SEQUENCE LISTING (5 sequences)

SEQ ID NO: 1 - RNA, 20 nt, Artificial Sequence
  chemically synthesized
cacgcgagccgaacgaacaa

SEQ ID NO: 2 - RNA, 22 nt, Artificial Sequence
  chemically synthesized
ucacgcgagccgaacgaacaaa

SEQ ID NO: 3 - DNA, 21 bp, Homo sapiens
cgcgagccgaacgaacaaatt

SEQ ID NO: 4 - RNA, 22 nt, Homo sapiens
uuuguucguucggcucgcguga

SEQ ID NO: 5 - RNA, 22 nt, Homo sapiens
uuuguucguucggcucgcguga

(19) United States
(12) Patent Application Publication
(10) Pub. No.: US 2018/0008641 A1
(43) Pub. Date: Jan. 11, 2018

(54) Use of MicroRNA 375 in Augmenting Stem Cell Based and Endogenous Ischemic Tissue Repair

(71) Applicant: Temple University-Of The Commonwealth System of Higher Education, Philadelphia, PA (US)

(72) Inventors: Raj Kishore, Devon, PA (US); Venkata Naga Srikanth Garikipati, Philadelphia, PA (US)

(21) Appl. No.: 15/527,767

(22) PCT Filed: Nov. 18, 2015
(86) PCT No.: PCT/US15/61230

§ 371 Date: May 18, 2017

Related U.S. Application Data
(60) Provisional application No. 62/081,279, filed on Nov. 18, 2014.

(51) Int. Cl. A61K 35/28; A61K 38/20; C12P 19/34; A61K 48/00

(52) U.S. Cl. CPC A61K 35/28; C12P 19/34; A61K 38/2066; A61K 48/00

(57) ABSTRACT

The present invention relates to compositions and methods useful for treating ischemic injury. In one embodiment, the present invention uses an inhibitor of miR-375 in a cell to enhance its survival.

### CROSS-REFERENCE TO RELATED APPLICATION

This application claims priority to U.S. Provisional Application Ser. No. 62/081,279 filed Nov. 18, 2014, the contents of which is incorporated by reference herein in its entirety.

### STATEMENT REGARDING FEDERALLY SPONSORED RESEARCH OR DEVELOPMENT

This invention was made with government support under HL091983 awarded by the National Institutes of Health (NIH). The government has certain rights in the invention.

### BACKGROUND OF THE INVENTION

In the absence of effective endogenous repair mechanisms after cardiac injury, cell-based therapies have rapidly emerged as a potential novel therapeutic approach in ischemic heart disease. After initial characterization of putative bone marrow-derived angiogenic progenitor cells (BMPAC) or endothelial progenitor cells (EPC) and their potential to promote cardiac neovascularization and to attenuate ischemic injury, a decade of intense preclinical research led to BMPAC-based clinical trials which yielded promising yet modest results (Assumus et al., 2002, Circulation 106:3009-17; Losordo et al., 2011, Circ Res 109:428-36; Strauer et al., 2002, Cirulcation 106:1913-8; Losordo et al., 2007, Circulation 115:3165-72; Kandala et al., 2013, Am J Cardiol 112:217-25). Preclinical studies suggest that microenvironment in the infarcted myocardium, including inflammation and oxidative damage, has adverse effects on transplanted stem cell survival and function (Grisar et al., 2005, Circulation 111:204-11; Werner and Nickenig, 2006, Arterioscl Throm Vasc Biol 26:257-66). Thus, enhancing survival and function of transplanted stem remains a recognized challenge.

Therefore, there is a long-felt need in the art for a therapeutic method of enhancing cell survival by which the cells mediates myocardial repair. The present invention fills this need.

### SUMMARY OF THE INVENTION

The present invention provides a composition for treating ischemic injury.

In one embodiment, the composition comprises an inhibitor of microRNA (miR)-375.

In one embodiment, the inhibitor of miR-375 is an oligonucleotide. In another embodiment oligonucleotide comprises at least one locked nucleic acid. In one embodiment the oligonucleotide comprises SEQ ID NO. 1. In another embodiment, the oligonucleotide comprises SEQ ID NO. 2.

In one embodiment, the composition further comprises IL-10.

In one embodiment, the composition further comprises a pharmaceutically acceptable carrier.

The present invention also provides a method of treating an ischemic heart in a subject.

In one embodiment, the method comprises isolating a stem cell from a subject; inhibiting miR-375 in the isolated stem cell to generate a miR-375 inhibited stem cell; and administering the miR-375 inhibited stem cell to the subject.

In one embodiment, the stem cell is a bone marrow-derived angiogenic progenitor cell (BMAPC).

In one embodiment inhibiting miR-375 in the stem cell further comprises administering to the cell an effective amount of an inhibitor of miR-375.

In one embodiment, the inhibitor of miR-375 is an oligonucleotide. In another embodiment oligonucleotide comprises at least one locked nucleic acid. In one embodiment the oligonucleotide comprises SEQ ID NO. 1. In another embodiment, the oligonucleotide comprises SEQ ID NO. 2.

In one embodiment, the method further comprises administering to the subject an effective amount of IL-10.

In one embodiment, administering the stem cell to the subject further comprises administering the stem cell to the subject through a route, the route selected from the group consisting of parenteral, intravenous, intraperitoneal, and bolus injection to a target tissue.

In one embodiment, the target tissue is cardiac tissue.

The present invention also provides a method of enhancing cell survival.

In one embodiment, the method comprises administering to a cell an effective amount of an inhibitor of miR-375.

### BRIEF DESCRIPTION OF THE DRAWINGS

FIG. 1 depicts results of experiments showing inflammatory stimulus enhances the expression of a number of microRNAs (miRs) in BMPAC. miRNA expression was normalized to U6 snRNA.

FIG. 2, comprising FIGS. 2A-2D, depicts results of experiments showing IL-10 regulates microRNA-375 (miR-375) expression in BMPACs. FIG. 2A depicts IL-10 inhibits myocardial infarction (MI)-induced myocardial miR-375 expression. ***, p<0.001 versus sham injured hearts; ##, p<0.01 versus MI injured hearts (n=4). FIG. 2B depicts wild-type (WT)-BMPAC/IL-10 KO BMPAC were stimulated with lipopolysaccharide (LPS), with LPS+IL-10 or IL-10 alone. Expression of miR-375 was measured by reverse transcriptase polymerase chain reaction (RT-PCR). FIG. 2C depicts BMPAC were subjected to hypoxia, hypoxia+IL-10, or IL-10 alone and miR-375 expression was measured by RT-PCR normalized to U6 with or without IL-10.n=3. ***, p<0.001 versus WT-Ctrl BMPAC; $$$, p<0.001 versus WT/IL-10 KO BMPAC+LPS. FIG. 2D depicts BMPACs were treated with LPS or IL-10 or both before the addition of 5 μg actinomycin D (Act-D). BMPACs were harvested 30, 60, and 120 minutes after the addition of Act-D (time 0), qRT-PCR was performed for miR-375. Expression data are expressed as percent of miR-375 remaining at each time point versus miR-375 levels at time 0. miR expression was normalized to U6 snRNA. **, p<0.01 ***, p<0.001 versus LPS alone; ##. p<0.01###. p<0.001 versus LPS+Act-D.

FIG. 3 depicts results of experiments where BMPACs were transfected with scrambled or antagomiR-375 (30 nM) for 24 h. Relative quantification of miR-375 levels normalized to U6 snRNA. ***P<0.001 Vs Scrambled BMPAC.

FIG. 4, comprising FIGS. 4A through 4E, depicts results of experiments showing Knockdown of microRNA-375 (miR-375) enhances WT BMPAC functions and rescues IL-10 KO BMPAC phenotype. FIG. 4A depicts representative photomicrographs (10×, scale bar=20 μM) of tube formation by matrigel angiogenesis assays in WT-BMPAC/IL-10 KO BMPAC transfected with scrambled or anti-miR-375. FIG. 4B depicts relative quantification of branch points. FIG. 4C depicts quantification of percentage of terminal deoxynucleotidyl transferase-mediated dUTP nick end-labeling (TUNEL+) cells were measured by fluorescence microscopy after apoptotic stimuli of 100 μmol/l H2O2 after WT-BMPAC/IL-10 KO BMPAC transfected with scrambled or anti-miR-375. FIG. 4D depicts quantification of apoptosis by caspase 3/7 assay after WT-BMPAC/IL-10 KO BMPAC transfected with scrambled or anti-miR-375. FIG. 4E depicts quantification of apoptosis by ciQuant assay after WT-BMPAC/IL-10 KO BMPAC transfected with scrambled or anti-miR-375. Results are presented as SEM for three independent experiments. ***, p<0.001; **, p<0.01; *, p<0.05 versus WT Scrambled BMPAC; ###, p<0.001; ##, p<0.01; #, p<0.05 versus IL-KO scrambled BMPAC.

FIG. 5 depicts results of experiments showing knockdown of miR-375 enhances WT BMPAC apoptosis and rescues IL-10-null BMPAC phenotype. Shown are representative Tunel staining of apoptotic nuclei (red) and DAPI (blue) after WT-BMPAC/IL-10 KO BMPAC were treated with scrambled or antagomiR-375 and subjected to H202 insult. n=3. ***P<0.001, **P<0.001 Vs WT Scrambled BMPAC; #p<0.05, ##p<0.01, ###p<0.001 Vs IL-KO scrambled BMPAC.

FIG. 6 depicts results of experiments showing knockdown of miR-375 enhances BMPAC proliferation. Quantification of optical density by MTT assay after WT-BMPAC/IL-10 KO BMPAC were treated with scrambled or antagomiR-375 for 24 h. n=3. ***P<0.001, **P<0.001 Vs WT Scrambled BMPAC; ###p<0.001 Vs IL-KO scrambled BMPAC.

FIG. 7 depicts results of experiments showing over expression of miR-375 induce apoptosis and reduces tube formation ability in WT/IL-10 KO BMPAC. BMPACs were transfected with scrambled or pre-miR-375 (30 nM) for 24 h. Relative quantification of miR-375 levels normalized to U6 snRNA.

FIG. 8, comprising FIGS. 8A through 8D, depicts results of experiments showing overexpression of miR-375 in BMPACs are susceptible to apoptosis and exhibit reduced tube formation ability. FIG. 8A depicts representative apoptotic nuclei (red) and DAPI (blue) nuclei stain by Tunel assay after WT-BMPAC/IL-10 KO BMPAC were treated with scrambled or pre-miR-375 and subjected to H202 insult. FIG. 8B depicts quantification of apoptosis by Tunel assay. FIG. 8C depicts representative photomicrographs of tube formation by matrigel angiogenesis assays in WT-BMPAC/IL-10 KO BMPAC transfected with scrambled or pre-miR-375. FIG. 8D depicts relative quantification of branch points, n=3. ***P<0.001, **P<0.001 Vs WT Scrambled BMPAC; #p<0.05, ##p<0.01, ###p<0.001 Vs IL-KO scrambled BMPAC.

FIG. 9, comprising FIGS. 9A through 9E, depicts results of experiments showing miR-375 directly targets PDK-1. FIG. 9A depicts relative mRNA expression of PDK-1 normalized to 18S. FIG. 9B depicts a representative immune-blot of PDK-1. FIG. 9C depicts relative quantification of PDK-1 protein. FIG. 9D depicts the study of the interaction between miR-375 and 3′UTR of PDK-1 mRNA by luciferase assay. FIG. 9E depicts the overexpression of miR-375 attenuates AKT phosphorylation. Results are presented as s.e.m for three independent experiments. n=3. ***P<0.00, **P<0.01, *P<0.05 Vs WT Scrambled BMPACs.

FIG. 10, comprising FIGS. 10A through 10D, depicts results of experiments showing down-regulation of PDK-1 inhibits the effects of anti-miR-375 on HUVECs apoptosis and tube formation. HUVECs were transfected with NC-siRNA or PDK-1 for 24 h. FIG. 10A depicts representative PDK-1 protein levels. FIG. 10B depicts Quantification of PDK-1 levels normalized to β-actin in HUVECs transfected with NC-siRNA, anti-miR-375, PDK-1 siRNA, PDK-1 siRNA+anti miR-375. FIG. 10C depicts quantification of apoptosis by tunel assay. FIG. 10D depicts relative quantification of branch points, Results are presented as s.e.m for three independent experiments. n=3. ***p<0.001Vs Scrambled ctrl HUVECs. ### P<0.001 Vs anti miR-375 treated HUVECs.

FIG. 11 depicts results of experiments showing GFP-lentiviral transduction of BMPAC. Shown are representative immunofluorescence image of GFP lentivirus infected BMPACs (10×, 100 um) in bright field, GFP (Green) BMPAC and the overlay of bright field and GFP BMPAC.

FIG. 12, comprising FIGS. 12A through 12G, depicts results of experiments showing increased survival of miR-375 Knockdown BMPACs in situ in the heart following myocardial infarction. BMPAC retention and survival in the myocardium is shown at 5 days after MI in anti-miR-375 BMPAC or scrambled BMPAC treated mice. Tunel staining detects apoptosis (red) of BMPAC (GFP-positive, green florescence) and DAPI (blue) for nuclear staining. Inset is images are higher magnification of yellow-boxed area. Arrows indicate GFP+TUNEL+ cells (40×, Scale bar 100 μm). (red), DAPI (blue) for nuclei staining. Inset is higher magnification of yellow-boxed area. Arrows indicate GFP+BrdU+ cells. FIG. 12A depicts image quantification of GFP+(BMPAC) at 5 days post-MI. FIG. 12B depicts graphical quantification of GFP+(BMPAC) at 5 days post-MI. FIG. 12C depicts quantitative analysis of GFP/TUNEL double positive cells at 5 days after MI. FIG. 12D depicts Increased GFP+BrdU+ cells (40×, scale bar=100 μm) within hearts treated with anti-miR-375 BMPAC compared scrambled BMPAC-treated hearts stained with green fluorescent protein (GFP; green), BrdU (red), DAPI (blue) for nuclei staining. Inset is higher magnification of yellow-boxed area. Arrows indicate GFP+BrdU+ cells. FIG. 12E depicts quantification of BrdU+/GFP+ cells in mice treated with anti-miR-375 BMPAC and Scrambled BMPAC (n=5). FIG. 12F depicts representative TUNEL staining image for cardiomyocyte apoptosis (green nuclei), alpha actinin (red), DAPI (blue) in border zone of LV infarct at 5 days post-MI. FIG. 12G depicts quantitative analysis of TUNEL+cardiomyocytes at 5 d post-MI. n=5 per group. ***, p<0.001; **, p<0.01 versus scrambled wild-type-BMPAC-treated groups.

FIG. 13, comprising FIGS. 13A through 13C, depicts results of experiments showing PDK-1 is upregulated in the anti-microRNA-375 BMPACs transplanted hearts after MI. FIG. 13A depicts representative western blot for PDK-1, pAKT, and total AKT protein expression in LV at 5 d post-MI normalized to β-actin. n=5 per group. FIG. 13B depicts quantification of PDK-1 relative to β-actin. FIG. 13C depicts quantification of P-AKT relative to AKT.

FIG. 14, comprising FIGS. 14A through 14F, depicts results of experiments showing transplantation of miR-375 knockdown BMPACs reduces fibrosis and enhances neovascularization and LV functional recovery 4 weeks after myocardial infarction. FIG. 14A depicts representative masons trichome stained heart (1×, scale bar=100 μm) treated with saline or wild-type (WT)-BMPAC treated with scrambled or anti-miR-375. FIG. 14b depicts quantitative analysis of infarct size (% LV area). FIG. 14C depicts representative immunofluorescence (20×, scale bar=100 μm) capillaries images taken within the infarct border zone of mice treated with saline or WT-BMPAC treated with scrambled or anti-miR-375. Capillaries were stained with BS-lectin-Alexa-555 (red) and nuclei were counterstained with 4′,6-diamidino-2-phenylindole (DAPI) (blue). FIG. 14D depicts quantification of border zone capillary number across treatments presented as the number of isolectin B4-positive capillaries and DAPI-stained nuclei per low-power visual fields (LPF). FIG. 14E depicts the % ejection fraction of mice receiving miR-375 knockdown WT-BMPAC. FIG. 14F depicts the % fractional shortening of mice receiving miR-375 knockdown WT-BMPAC.

FIG. 15, comprising FIGS. 15A through 15F, depicts results of experiments showing transplantation of miR-375 Knockdown IL-10 KO BMPACs partially attenuate left ventricular remodeling after MI. FIG. 15A depicts representative masons trichome stained heart (28 d post MI) treated with scrambled or anti-miR-375 to IL-10 KO BMPACs. FIG. 15B depicts quantitation of infarct size. FIG. 15C depicts representative immunofluorescence capillaries images taken within the infarct border zone of mice (28 d post MI) treated with scrambled or anti-miR-375 IL-10 KO BMPACs. FIG. 15D depicts quantitation of lectin counts/LPF. FIG. 15E depicts quantitation of ejection fraction. FIG. 15F depicts quantitation of fractional shortening

FIG. 16, comprising FIGS. 16A and 16B, depicts results of experiments showing anti-miR-375 BMPAC conditioned medium reduces cardiomyocyte apoptosis in vitro. FIG. 16A depicts representative apoptotic nuclei (red) and DAPI (blue) nuclei stain by Tunel assay after NRVM were treated with scrambled or anti-miR-375 supernatant, subjected to H202 insult. FIG. 16B depicts quantification of apoptosis by Tunel assay.

FIG. 17, comprising FIGS. 17A through 17E, depicts results of experiments showing anti-miR-375 BMPAC transplantation enhances paracrine activity in vivo. qPCR was used to analyze mRNA expression of angiogenic molecules in the border zone of LV infarct at 28 d post-MI in saline or BMPAC ctrl or BMPAC anti miR-375 groups. The mRNA expression was normalized to 18S expression. FIG. 17A depicts quantitative real-time PCR analysis of mRNA expression of angiogenic molecule VEGF. FIG. 17B depicts quantitative real-time PCR analysis of mRNA expression of angiogenic molecule IGF-1. FIG. 17C depicts quantitative real-time PCR analysis of mRNA expression of angiogenic molecule Ang-1. FIG. 17D depicts quantitative real-time PCR analysis of mRNA expression of angiogenic molecule SDF-1. FIG. 17E depicts quantitative real-time PCR analysis of mRNA expression of angiogenic molecule HGF.

FIG. 18 depicts a flow chart demonstrating the role of microRNA-375 BMPAC mediated cardiac regeneration. BMPACs inhibits IL-10 regulated miR-375 leading to activation of PDK-1/AKT signaling, PDK-1 (target of miR-375), thereby enhancing the neovascularization and also BMPAC survival post transplantation in myocardial infarction mice.

FIG. 19 depicts results of experiments showing miR-375 is elevated in the human heart failure patients. Shown is quantitative real-time PCR analysis of miR-375 expression in the border zone of\ LV infarct at 5 d post-MI. n=5/group.

FIG. 20, comprising FIGS. 20A through 20C, depicts results of experiments showing myocardial miR-375 knockdown inhibits post-MI LV inflammatory cell migration. FIG. 20A depicts quantitative real-time PCR analysis of miR-375 expression in the border zone of\ LV infarct at 5 d post-MI. n=5/group. FIG. 20B depicts Immunofluorescent staining (20×, Scale bar 100 μm) of inflammatory cells (CD68+, red) in the border zone of infarct at 5 d post-MI. FIG. 20C depicts quantitative analysis of infiltrating CD68+ cells per LPF at 5 d post-MI. n=5/group.

FIG. 21, comprising FIGS. 21A and 21B, depicts results of experiments showing myocardial miR-375 knockdown inhibits post-MI inflammatory cytokines expression. FIG. 21A depicts quantitative real-time PCR analysis of miR-375 expression in the border zone of\ LV infarct at 5 d post-MI. n=5/group. FIG. 21B depicts quantitative cytokine array analysis of pro-inflammatory cytokines in the border zone of LV infarct at 5 d post-MI.

FIG. 22, comprising FIGS. 22A and 22B, depicts results of experiments showing myocardial miR-375 knockdown Attenuates Post-MI LV Dysfunction. FIG. 22A depicts representative echocardiography % ejection fraction analysis shown in bars, in the hearts treated with scrambled or LNA anti-miR-375. FIG. 22B depicts representative echocardiography % fractional shortening analysis shown in bars, in the hearts treated with scrambled or LNA anti-miR-375.

FIG. 23, comprising FIGS. 23A through 23D, depicts results of experiments showing myocardial miR-375 knockdown reduces fibrosis after MI and enhances neovascularization after MI. FIG. 23A depicts representative masons trichome stained heart (28 d post MI) treated with scrambled or LNA anti-miR-375. FIG. 23B depicts quantitative analysis of fibrosis area (% LV area) at 28 d post-MI after saline. (n=5/each group). FIG. 23C depicts representative immunofluorescence capillaries images taken within the infarct border zone of mice (28 d post MI) treated with scrambled or LNA anti-miR-375. Capillaries were stained with BS-lectin-Alexa-555 (red) and nuclei were counterstained with DAPI (blue). (40×, Scale bar 100 μm). FIG. 23D depicts quantification of border zone capillary number across treatments presented as the number of isolectin B4-positive capillaries and DAPI-stained nuclei per high power field (LPF) (n=5/each group).

FIG. 24, comprising FIGS. 24A through 24C, depicts results of experiments showing LNA anti-miR-375 targets PDK-1 in the MI heart. FIG. 24A depicts representative western blots of PDK-1, pAKT and total AKT protein expression in LV at 5 d post-MI. Equal loading of proteins in each lane is shown by β-actin. FIG. 24B depicts quantitation of PDK-1 levels in LV at 5 d post-MI. FIG. 24C depicts quantitation of pAKT levels in LV at 5 d post-MI.

### DETAILED DESCRIPTION

The present invention is based, in part, on the discovery that IL-10 regulates microRNA-375 (miR-375) signaling in BMPACs to enhance their survival and function in ischemic myocardium after MI and attenuates left ventricular dysfunction after MI. For example, it is demonstrated herein that miR-375 expression is significantly upregulated in BMPACs upon exposure to inflammatory/hypoxic stimulus and also after MI. IL-10 knockout mice displayed significantly elevated miR-375 levels. Ex vivo miR-375 knockdown in BMPAC before transplantation in the ischemic myocardium after MI significantly improved the survival and retention of transplanted BMPACs and also BMPAC-mediated post-infarct repair, neovascularization, and LV functions. In vitro studies revealed that knockdown of miR-375-enhanced BMPAC proliferation and tube formation and inhibited apoptosis; over expression of miR-375 in BMPAC had opposite effects. Mechanistically, miR-375 negatively regulated 3-phosphoinositide-dependent protein kinase-1 (PDK-1) expression and PDK-1-mediated activation of PI3kinase/AKT signaling. BMPAC isolated from IL-10-deficient mice showed elevated basal levels of miR-375 and exhibited functional deficiencies, which were partly rescued by miR-375 knockdown, enhancing BMPAC function in vitro and in vivo. Therefore, without wishing to be bound by any particular theory, it is believed that miR-375 is negatively associated with BMPAC function and survival and IL-10-mediated repression of miR-375 enhances BMPAC survival and function. Thus, the present invention overcomes liabilities of limited survival and function of transplanted stem cells for ischemic tissue repair and regeneration.

In certain embodiments, the present invention provides compositions and methods for enhancing cell survival by inhibiting microRNA (miR or miRNA)-375 expression or activity. For example, in certain embodiments, the compositions and methods described herein are used to enhance stem-cell based therapies by improving the survival of administered stem cells.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, the phrase “cardiovascular condition, disease or disorder” is intended to include all disorders characterized by insufficient, undesired or abnormal cardiac function, e.g., ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results from an occlusive (e.g., thrombotic or embolic) event in the coronary circulation and produces an environment in which the myocardial metabolic demands exceed the supply of oxygen to the myocardial tissue.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

The term “derived from” is used herein to mean to originate from a specified source.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

“Expandability” is used herein to refer to the capacity of a cell to proliferate, for example, to expand in number or in the case of a cell population to undergo population doublings.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein, the term “growth medium” is meant to refer to a culture medium that promotes growth of cells. A growth medium will generally contain animal serum. In some instances, the growth medium may not contain animal serum.

An “isolated cell” refers to a cell which has been separated from other components and/or cells which naturally accompany the isolated cell in a tissue or mammal.

As used herein, the term “myocardial injury” or “injury to myocardium” refers to any structural or functional disorder, disease, or condition that affects the heart and/or blood vessels. Examples of myocardial injury can include, but are not limited to, arterial disease, atheroma, atherosclerosis, arteriosclerosis, coronary artery disease, arrhythmia, angina pectoris, congestive heart disease, ischemic cardiomyopathy, myocardial infarction, stroke, transient ischemic attack, aortic aneurysm, cardiopericarditis, infection, inflammation, valvular insufficiency, vascular clotting defects, and combinations thereof.

The terms “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew itself or to produce progeny cells which will differentiate into the desired cell type. Unlike pluripotent stem cells, lineage-committed progenitor cells are generally considered to be incapable of giving rise to numerous cell types that phenotypically differ from each other. Instead, progenitor cells give rise to one or possibly two lineage-committed cell types.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, a cell exists in a “purified form” when it has been isolated away from all other cells that exist in its native environment, but also when the proportion of that cell in a mixture of cells is greater than would be found in its native environment. Stated another way, a cell is considered to be in “purified form” when the population of cells in question represents an enriched population of the cell of interest, even if other cells and cell types are also present in the enriched population. A cell can be considered in purified form when it comprises in some embodiments at least about 10% of a mixed population of cells, in some embodiments at least about 20% of a mixed population of cells, in some embodiments at least about 25% of a mixed population of cells, in some embodiments at least about 30% of a mixed population of cells, in some embodiments at least about 40% of a mixed population of cells, in some embodiments at least about 50% of a mixed population of cells, in some embodiments at least about 60% of a mixed population of cells, in some embodiments at least about 70% of a mixed population of cells, in some embodiments at least about 75% of a mixed population of cells, in some embodiments at least about 80% of a mixed population of cells, in some embodiments at least about 90% of a mixed population of cells, in some embodiments at least about 95% of a mixed population of cells, and in some embodiments about 100% of a mixed population of cells, with the proviso that the cell comprises a greater percentage of the total cell population in the “purified” population that it did in the population prior to the purification. In this respect, the terms “purified” and “enriched” can be considered synonymous.

As used herein, “tissue engineering” refers to the process of generating tissues ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

The term “complementary” (or “complementarity”) refers to the specific base pairing of nucleotide bases in nucleic acids. The term “perfect complementarity” as used herein refers to complete (100%) complementarity within a contiguous region of double stranded nucleic acid, such as between a hexamer or heptamer seed sequence in an miRNA and its complementary sequence in a target polynucleotide, as described in greater detail herein.

An “antisense nucleic acid” (or “antisense oligonucleotide”) is a nucleic acid molecule (RNA or DNA) which is complementary to an mRNA transcript or a selected portion thereof. Antisense nucleic acids are designed to hybridize with the transcript and, by a variety of different mechanisms, prevent if from being translated into a protein; e.g., by blocking translation or by recruiting nucleic acid-degrading enzymes to the target mRNA.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Homologous, homology” or “identical, identity” as used herein, refer to comparisons among amino acid and nucleic acid sequences. When referring to nucleic acid molecules, “homology,” “identity,” or “percent identical” refers to the percent of the nucleotides of the subject nucleic acid sequence that have been matched to identical nucleotides by a sequence analysis program. Homology can be readily calculated by known methods. Nucleic acid sequences and amino acid sequences can be compared using computer programs that align the similar sequences of the nucleic or amino acids and thus define the differences. In preferred methodologies, the BLAST programs (NCBI) and parameters used therein are employed, and the DNAstar system (Madison, Wis.) is used to align sequence fragments of genomic DNA sequences. However, equivalent alignment assessments can be obtained through the use of any standard alignment software.

The term, “miR” or “miRNA” or “microRNA” is used herein in accordance with its ordinary meaning in the art. miRNAs are single-stranded RNA molecules of about 20-24 nucleotides, although shorter or longer miRNAs, e.g., between 18 and 26 nucleotides in length, have been reported. miRNAs are encoded by genes that are transcribed from DNA but not translated into protein (non-coding RNA), although some miRNAs are coded by sequences that overlap protein-coding genes. miRNAs are processed from primary transcripts known as pri-miRNA to short stem-loop structures called pre-miRNA and finally to functional miRNA. Mature miRNA molecules are partially complementary to one or more messenger RNA (mRNA) molecules, and they function to regulate gene expression.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression vector.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, the present invention provides compositions useful for enhancing cell survival. In one embodiment, the composition comprises an inhibitor of microRNA (miR or miRNA)-375. In one embodiment, the miR-375 inhibitor is an oligonucleotide. In one embodiment, the oligonucleotide comprises at least one locked nucleic acid. In another embodiment, the at least one locked nucleic acid comprises SEQ ID NO. 1. In yet another embodiment the oligonucleotide comprises SEQ ID NO. 2

In certain aspects, the composition further comprises another active agent. In one embodiment, the active agent includes, but is not limited to IL-10, genes targets of miR-375, and kinase targets of miR-375. In another embodiment, the active agent increases PDK-1 expression.

In some embodiments, the composition further comprises a pharmaceutically acceptable carrier.

In another aspect, the present invention provides a method of treating an ischemic heart in a subject, the method comprising isolating a cell from the subject; inhibiting miR-375 in the cell; and administering the cell to the subject.

In one embodiment, the cell is a stem cell. In another embodiment, the stem cell is a bone marrow-derived angiogenic progenitor cell (BMAPC). In another embodiment the stem cell is an endothelial progenitor cell (EPC).

In one embodiment, inhibiting miR-375 in the stem cell comprises administering to the cell an effective amount of an inhibitor of miR-375. In one embodiment, the cell is a stem cell. In another embodiment, the stem cell is a bone marrow-derived angiogenic progenitor cell (BMAPC). In yet another embodiment, the stem cell is a human stem cell.

In one embodiment the inhibitor of miR-375 includes, but is not limited to, an oligonucleotide. In one embodiment, the oligonucleotide inhibitor comprises at least one locked nucleic acid. In one embodiment, the at least one locked nucleic acid comprises SEQ ID NO. 1. In yet another embodiment the oligonucleotide comprises SEQ ID NO. 2.

In another embodiment, administering the stem cell to the subject further comprises administering the stem cell to the subject through a desired route. In some embodiments the route includes, but is not limited to, parenteral, intravenous, intraperitoneal, and bolus injection to a target tissue. In certain embodiments, the target tissue is cardiac tissue.

In one embodiment, the method further comprises administering to the subject an effective amount of an active agent. In one embodiment, the active agent but is not limited to IL-10, gene targets of miR-375, and kinase targets of miR-375.

In one embodiment, the method further comprises increasing phosphoinositide-dependent protein kinase-1 (PDK-1) expression.

In another aspect, the present invention also provides methods of treating a cardiovascular condition, disease or disorder in a subject, the method comprising isolating a stem cell from the subject; inhibiting miR-375 in the stem cell; and administering the stem cell to the subject.

In one embodiment the cardiovascular condition, disease or disorder includes, but is not limited to ischemic injury, heart failure, myocarditis and inflammatory diseases linked to increased miR375 expression.

In one embodiment, inhibiting miR-375 in the stem cell further comprises administering to the cell an effective amount of an inhibitor of miR-375. In one embodiment, the inhibitor of miR-375 includes, but is not limited to a locked nucleic acid (LNA) and an anti-miR. In another embodiment, the LNA comprises CACGCGAGCCGAACGAACAA (SEQ ID NO. 1). In yet another embodiment, the anti-miR comprise comprises UCACGCGAGCCGAACGAACAAA (SEQ ID NO. 2).

In one embodiment, the method further comprises reducing fibrosis. In another embodiment, the method further comprises enhancing neovascularization of the cell. In one embodiment, the stem cell is a bone marrow-derived angiogenic progenitor cell. In one embodiment, the subject is a mammal. In another embodiment, the subject is a human.

In yet another aspect, the present invention provides a method of enhancing cell survival. In one embodiment, the method comprises administering to the cell an effective amount of an inhibitor of miR-375.

### Composition

In one embodiment, the present invention provides a composition for enhancing cell survival. In another embodiment, the invention provides a composition for treating an ischemic heart in a subject. In one embodiment, the present invention provides a composition for enhancing cell survival and for treating an ischemic heart in a subject. In certain aspects, the composition comprises one or more inhibitors of miR-375. In some embodiments, the inhibitor of miR-375 is a nucleic acid. In certain embodiments the nucleic acid inhibitor of miR-375 includes, but is not limited to, a locked nucleic acid (LNA), an anti-miR, and the like. In other embodiments, the composition comprises an activator of PDK-1. In yet another embodiment, the composition comprises an inhibitor of miR-375 and IL-10 or a fragment thereof.

microRNA The discovery of microRNAs (miR) has opened a new approach regarding regulation of cellular processes such as proliferation, differentiation, cell metabolism, apoptosis, and angiogenesis (Bartel, 2009, Cell 136:215-33; Krol et al., 2010, Nat Rev Genet 11:597-610; Almeida et al., 2011, Mutat Res 717:1-8). BMPACs from IL-10 knockout (IL-10 KO) mice are functionally impaired (Krishnamurthy et al., 2011, Circ Res 109:1280-9). Mononuclear cells from IL-10 KO mice express high levels of miR-375 (Schaefer et al., 2011, J Immunol 187:5834-41). The miR-375 gene has been shown to be found on chromosome 2 in humans and chromosome 1 in mice and located in an intergenic region between the cryba2 (b-A2 crystallin, an eye lens component) and Ccdc108 (coiled-coil domain-containing protein 108) genes and is highly conserved between humans and mice (Yan et al., 2014, Int J Cancer 135:1011-8; Keller et al., 2007, J Biol Chem 282:32084-92; Baroukh and Obberghen, 2009, FEBS J 276:6509-21). Emerging evidence suggests an association of decreased miR-375 expression with tumorigenesis and progression in melanoma, carcinoma of the head and neck, esophageal, gastric, or prostate cancer (Yan et al., 2014, Int J Cancer 135:1011-8). Furthermore, patients with heart failure and/or diabetes have high levels of miR-375 (Akat et al., 2014, PNAS 111:11151-6; Zhao et al., 2010, Pancrease 39:843-6). However, no prior study has established a role of miR-375 either in BMPAC/stem cell biology or ischemic tissue repair.

Transcription of miRNA genes, including that of miR-375, is mediated by RNA polymerase II (pol II) to produce long primary transcripts (pri-miRNAs) that are often several kilobases long. Pri-miRNA transcripts contain both a 5′ terminal cap structure and a 3′ terminal poly(A) tail. Several poly(A)-containing transcripts containing both miRNA sequences and regions of adjacent mRNAs have been characterized.

The maturation of miRNA from pri-miRNAs involves trimming of pri-miRNAs into hairpin intermediates called precursor miRNAs (pre-miRNAs), that are subsequently cleaved into mature miRNAs. The stem-loop structure of pri-miRNA molecules are cleaved by the nuclear RNase III enzyme Drosha to release the pre-miRNA molecules. Drosha is a large protein of approximately 160 kDa, and, in humans, forms an even larger complex of approximately 650 kDa known as the Microprocessor complex. The enzyme is a Class II RNAse III enzyme having a double-stranded RNA binding domain (dsRBD).

Following export of pre-miRNA molecules to the cytoplasm, another RNase III enzyme called “Dicer” cleaves the pre-miRNA to produce the mature miRNA. Mature miRNAs are incorporated into an effector complex known as the miRNA-containing RNA-induced silencing complex or miRISC.

Additional information concerning miRNAs and associated pri-miRNA and pre-miRNA sequences is available in miRNA databases such as miRBase (Griffiths-Jones et al. 2008 Nucl Acids Res 36, (Database Issue:D154-D158) and the NCBI human genome database.

Human miR-375 has a precursor sequence of SEQ ID NO. 3 a mature sequence of SEQ ID NO. 4 and is listed under GenBank accession number NR 029867. Homologous miR-375 genes of non-human species are also known, including for example those available in GenBank. Examples include, but are not limited to, those listed under GenBank accession numbers NR 029876 (from Mus musculus); NR 034295 (from Apis mellifera); NR 032271 (from Rattus norvegicus); NR 036388 (from Strongylocentrotus purpuratus); NR 129967 (from Anolis carolinensis); and NR 049460 (from Canis lupus familiaris).

(SEQ ID NO. 3)
pre-miR-375 has the sense sequence
CGCGAGCCGAACGAACAAATT.
pre-miR-375 has the antisense sequence
(SEQ ID NO. 4)
UUUGUUCGUUCGGCUCGCGUGA.
(SEQ ID NO. 5)
miR-375 has the sequence UUUGUUCGUUCGGCUCGCGUGA.

IL-10

A cardio-protective role of anti-inflammatory cytokine interleukin-10 (IL-10) therapy has been established in mouse models of acute myocardial infarction (AMI) and pressure-overload, which in turn is mediated by signal transduction pathways including p38 MAP kinase, NF-kB, and STAT-3 (Krishnamurthy et al., 2010, FASEB J 24:2484-94; Krishnamurthy et al., 2009, Circ Res 104:e9-18; Verman et al., 2012, Cirulation 126:418-29; Jones et al., 2001, Am J Physiol Heart Circ Physiol 281:H48-52). Patients with high serum levels of proinflammatory cytokines have low circulating and functionally impaired BMPACs (Grisar et al., 2005, Circulation 111:204-11; Werner and Nickenig, 2006, Arterioscl Throm Vasc Biol 26:257-66). Combined therapy with bone marrow progenitor cells and IL-10 is more effective in the improvement of post-AMI left ventricular (LV) function than bone marrow progenitor cells alone (Schaefer et al., 2011, J Immunol 187:5834-41). Further role of IL-10 on BMPAC biology, signaling, and function has never been studied and warrants thorough investigation.

Inhibitors

In one embodiment, the present invention provides a composition for treating or preventing a disease or disorder associated with an increase in miR-375. In certain embodiments, the disease or disorder is ischemic injury. In certain embodiments, the composition inhibits the expression, activity, or both of miR-375.

In one embodiment, the composition of the invention comprises an inhibitor of miR-375. An inhibitor of miR-375 is any compound, molecule, or agent that reduces, inhibits, or prevents the function of miR-375. For example, an inhibitor of miR-375 is any compound, molecule, or agent that reduces miR-375 expression, activity, or both. In one embodiment, an inhibitor of miR-375 comprises a nucleic acid, a peptide, a small molecule, a siRNA, a ribozyme, an antisense nucleic acid, an antagonist, an aptamer, a peptidomimetic, or any combination thereof.

Nucleic Acid Inhibitors

In some aspects, the invention includes an isolated nucleic acid or an isolated oligonucleotide. In some instances the inhibitor is an siRNA or antisense molecule, which inhibits miR-375. In one embodiment, the nucleic acid comprises a promoter/regulatory sequence such that the nucleic acid is preferably capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In one embodiment, siRNA is used to decrease the level of miR-375. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of IL-18 or IL-18R using RNAi technology.

In another aspect, the invention includes a vector comprising an siRNA or antisense polynucleotide. Preferably, the siRNA or antisense polynucleotide is capable of inhibiting the expression of a target polypeptide, wherein the target polypeptide is selected from the group consisting of p21 and telomerase. The incorporation of a desired polynucleotide into a vector and the choice of vectors is well-known in the art as described in, for example, Sambrook et al. (2012), and in Ausubel et al. (1997), and elsewhere herein.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In some embodiments, oligonucleotides useful for inhibiting the activity of miRNAs are about 5 to about 25 nucleotides in length, about 10 to about 30 nucleotides in length, or about 20 to about 25 nucleotides in length. In certain embodiments, oligonucleotides targeting miRNAs are about 8 to about 18 nucleotides in length, in other embodiments about 12 to about 16 nucleotides in length, and in other embodiments about 7-8 nucleotides in length. Any 7-mer or longer complementary to a target miRNA may be used, that is, any anti-miR complementary to the 5′ end of the target miRNA and progressing across the full complementary sequence of the target miRNA.

Oligonucleotides can comprise a sequence that is at least partially complementary to a target miRNA sequence, for example, at least about 75%, 80%, 85%, 90%, 95%, 96%, 97%, 98%, or 99% complementary to a target miRNA sequence. In some embodiments, the oligonucleotide can be substantially complementary to a target miRNA sequence, that is at least about 90%, 95%, 96%, 97%, 98%, or 99% complementary to a target polynucleotide sequence. In one embodiment, the oligonucleotide comprises a sequence that is 100% complementary to a target miRNA sequence. In some embodiments, the target miRNA is miRNA-375 or pre-miR-375.

In some embodiments, the oligonucleotides are anti-miRs. Anti-miRs are single-stranded, chemically-modified ribonucleotides that are at least partially complementary to miRNAs and therefore may silence them. See, for example, Krutzfeldt, et al. (2005, Nature 438:685-9). Anti-miRs may comprise one or more modified nucleotides, such as 2′-O-methyl-sugar modifications. In some embodiments, anti-miRs comprise only modified nucleotides. Anti-miRs can also comprise one or more phosphorothioate linkages resulting in a partial or full phosphorothioate backbone. To facilitate in vivo delivery and stability, the anti-miR can be linked to a cholesterol or other moiety at its 3′ end. Anti-miRs suitable for inhibiting can be about 15 to about 50 nucleotides in length, about 18 to about 30 nucleotides in length, and about 20 to about 25 nucleotides in length. The anti-miRs can be at least about 75%, 80%, 85%, 90%, 95%, 96%, 97%, 98%, or 99% complementary to the target miRNA sequence. In some embodiments, the anti-miR may be substantially complementary to a target miRNA sequence, that is at least about 95%, 96%, 97%, 98%, or 99% complementary to a target polynucleotide sequence. In other embodiments, the anti-miRs are 100% complementary to a target miRNA sequence.

Oligonucleotides or anti-miRs may comprise a sequence that is substantially complementary to a precursor miRNA sequence (pre-miRNA) or primary miRNA sequence (pri-miRNA) of a miRNA. In some embodiments, the oligonucleotide comprises a sequence that is located outside the 3′-untranslated region of a target of that miRNA. In some embodiments, the oligonucleotide comprises a sequence that is located inside the 3′-untranslated region of a target of that miRNA.

Locked Nucleic Acids

In some embodiments, the oligonucleotide of the invention comprises locked nucleic acids (LNAs). LNAs are modified nucleotides or ribonucleotides that contain an extra bridge between the 2′ and 4′ carbons of the ribose sugar moiety resulting in a “locked” conformation, and/or bicyclic structure. In one embodiment, the oligonucleotide contains one or more LNAs. Examples of locked nucleotides that can be incorporated in the oligonucleotides of the invention include those described in U.S. Pat. No. 6,403,566 and U.S. Pat. No. 6,833,361, both of which are hereby incorporated by reference in their entireties.

In certain embodiments, the oligonucleotide comprising locked nucleic acids comprises the sequence SEQ ID NO. 1.

Small Molecule Inhibitors

In various embodiments, the inhibitor is a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In one embodiment, the small molecule inhibitor of the invention comprises an analog or derivative of an inhibitor described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any of a small molecule inhibitor in accordance with the present invention can be used to reduce skin pigmentation.

In one embodiment, the small molecule inhibitors described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

Polypeptide Inhibitors

In other related aspects, the invention includes an isolated peptide inhibitor that inhibits miR-375. For example, in one embodiment, the peptide inhibitor of the invention inhibits miR-375 directly by binding to miR-375 thereby preventing the normal functional activity of miR-375.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

Antibody Inhibitors

The invention also contemplates an inhibitor of miR-375 comprising an antibody, or antibody fragment, specific for miR-375. That is, the antibody can inhibit miR-375 to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain F_{V} molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

Activators

In certain embodiments, the composition comprises an activator of PDK-1, which is a direct target of miR-375. In certain embodiments, the activator of PDK-1 provides an angiogenic and anti-apoptotic benefit. It will be understood by one skilled in the art, based upon the disclosure provided herein, that an increase in the level of PDK-1 encompasses the increase in PDK-1 expression, including transcription, translation, or both. The skilled artisan will also appreciate, once armed with the teachings of the present invention, that an increase in the level of PDK-1 includes an increase in PDK-1 activity (e.g., enzymatic activity, substrate binding activity, etc.). Thus, increasing the level or activity of PDK-1 includes, but is not limited to, increasing the amount of PDK-1 polypeptide, and increasing transcription, translation, or both, of a nucleic acid encoding PDK-1; and it also includes increasing any activity of a PDK-1 polypeptide as well.

The increased level or increased activity of PDK-1 can be assessed using a wide variety of methods, including those disclosed herein, as well as methods well-known in the art or to be developed in the future. That is, the skilled artisan would appreciate, based upon the disclosure provided herein, that increasing the level or activity of PDK-1 can be readily assessed using methods that assess the level of a nucleic acid encoding PDK-1 (e.g., mRNA), the level of PDK-1 polypeptide, and/or the level of PDK-1 activity in a biological sample obtained from a subject.

One of skill in the art will realize that in addition to activating PDK-1 directly, diminishing the amount or activity of a molecule that itself diminishes the amount or activity of PDK-1 can serve to increase the amount or activity of PDK-1. Thus, a PDK-1 activator can include, but should not be construed as being limited to, a chemical compound, a protein, a peptidomemetic, an antibody, a ribozyme, and an antisense nucleic acid molecule. One of skill in the art would readily appreciate, based on the disclosure provided herein, that a PDK-1 activator encompasses a chemical compound that increases the level, enzymatic activity, or substrate binding activity of PDK-1. Additionally, a PDK-1 activator encompasses a chemically modified compound, and derivatives, as is well known to one of skill in the chemical arts.

The PDK-1 activator compositions and methods of the invention that increase the level or activity (e.g., enzymatic activity, substrate binding activity, etc.) of PDK-1 include activating antibodies. The activating antibodies of the invention include a variety of forms of antibodies including, for example, polyclonal antibodies, monoclonal antibodies, intracellular antibodies (“intrabodies”), Fv, Fab and F(ab)2, single chain antibodies (scFv), heavy chain antibodies (such as camelid antibodies), synthetic antibodies, chimeric antibodies, and a humanized antibodies. In one embodiment, the activating antibody of the invention is an antibody that specifically binds to PDK-1.

Further, one of skill in the art would, when equipped with this disclosure and the methods exemplified herein, appreciate that a PDK-1 activator includes such activators as discovered in the future, as can be identified by well-known criteria in the art of pharmacology, such as the physiological results of activation of PDK-1 as described in detail herein and/or as known in the art. Therefore, the present invention is not limited in any way to any particular PDK-1 activator as exemplified or disclosed herein; rather, the invention encompasses those activators that would be understood by the routineer to be useful as are known in the art and as are discovered in the future.

Further methods of identifying and producing a PDK-1 activator are well known to those of ordinary skill in the art, including, but not limited, obtaining an activator from a naturally occurring source (e.g., Streptomyces sp., Pseudomonas sp., Stylotella aurantium, etc.). Alternatively, a PDK-1 activator can be synthesized chemically. Further, the routineer would appreciate, based upon the teachings provided herein, that a PDK-1 activator can be obtained from a recombinant organism. Compositions and methods for chemically synthesizing PDK-1 activators and for obtaining them from natural sources are well known in the art and are described in the art.

One of skill in the art will appreciate that an activator can be administered as a small molecule chemical, a protein, an antibody, a nucleic acid construct encoding a protein, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an activator of PDK-1. (Sambrook et al., 2012, Molecular Cloning: A Laboratory Manual, Cold Spring Harbor Laboratory, New York; Ausubel et al., 1997, Current Protocols in Molecular Biology, John Wiley & Sons, New York).

One of skill in the art will realize that diminishing the amount or activity of a molecule that itself diminishes the amount or activity of PDK-1 can serve to increase the amount or activity of PDK-1. Antisense oligonucleotides are DNA or RNA molecules that are complementary to some portion of an mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of antisense oligonucleotide to diminish the amount of a molecule that causes a decrease in the amount or activity of PDK-1, thereby increasing the amount or activity of PDK-1. Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing a protein that diminishes the level or activity of PDK-1 can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

Nucleic Acids and Vectors

In one embodiment, the methods and compositions of the invention comprise an isolated nucleic acid. For example, in one embodiment, the composition comprises at least one inhibitor of miR-375, wherein the inhibitor is an isolated nucleic acid. In one embodiment, the isolated nucleic acid is an anti-miR. In another embodiment, the isolated nucleic acid is a LNA.

The isolated nucleic acid may comprise any type of nucleic acid, including, but not limited to DNA and RNA. For example, in one embodiment, the composition comprises an isolated DNA molecule, including for example, an isolated cDNA molecule, encoding an anti-miR or LNA of the invention, or functional fragment thereof. In one embodiment, the composition comprises an isolated RNA molecule comprising an anti-miR or LNA of the invention, or a functional fragment thereof. The isolated nucleic acids may be synthesized using any method known in the art.

The present invention also includes a vector in which the isolated nucleic acid of the present invention is inserted. The art is replete with suitable vectors that are useful in the present invention. Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

In brief summary, the expression of natural or synthetic nucleic acids encoding an RNA and/or peptide is typically achieved by operably linking a nucleic acid encoding the RNA and/or peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated.

In certain embodiments, the vector also includes conventional control elements which are operably linked to the transgene in a manner which permits its transcription, translation and/or expression in a cell transfected with the plasmid vector or infected with the virus produced by the invention. As used herein, “operably locked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

The selection of appropriate promoters can readily be accomplished. In certain aspects, one would use a high expression promoter. One example of a suitable promoter is the immediate early cytomegalovirus (CMV) promoter sequence. This promoter sequence is a strong constitutive promoter sequence capable of driving high levels of expression of any polynucleotide sequence operatively linked thereto. The Rous sarcoma virus (RSV) and MMT promoters may also be used. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication.

Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of the nucleic acid and/or protein, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, nanoparticles, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular protein, e.g., by immunological means (ELISAs and Western blots) or by assays described herein to identify agents falling within the scope of the invention.

In certain embodiments, the composition comprises a cell genetically modified to express one or more isolated nucleic acids and/or proteins described herein. For example, the cell may be transfected or transformed with one or more vectors comprising a nucleic acid encoding an anti-miR or a LNA. The cell can be the subject's cells or they can be haplotype matched. In specific embodiments, the cell is a stem cell. In some embodiments the stem cell is a BMAPC.

Scaffolds

The present invention provides a scaffold or substrate composition comprising a cell, an inhibitor of the invention, an activator of the invention, a peptide of the invention or any combination thereof. In some embodiments, the cell is a stem cell. In other embodiments, the cell is a BMAPC.

For example in one embodiment, the scaffold or substrate composition comprising an inhibitor of miR-375, PDK-1, a PDK-1-derived peptide, a nucleic acid molecule encoding PDK-1 or PDK-1 peptide, a cell producing PDK-1 or PDK-1 peptide, a BMAPC or BMAPC progenitor cell, or a combination thereof.

For example, in one embodiment, an inhibitor of miR-375, PDK-1, a PDK-1-derived peptide, a nucleic acid molecule encoding PDK-1 or PDK-1 peptide, a cell producing PDK-1 or PDK-1 peptide, a BMAPC or BMAPC progenitor cell, or a combination thereof is within a scaffold.

In another embodiment inhibitor of miR-375, PDK-1, a PDK-1-derived peptide, a nucleic acid molecule encoding PDK-1 or PDK-1 peptide, a cell producing PDK-1 or PDK-1 peptide, a BMAPC or BMAPC progenitor cell, or a combination thereof is applied to the surface of a scaffold.

The scaffold of the invention may be of any type known in the art. Non-limiting examples of such a scaffold includes a, hydrogel, electrospun scaffold, foam, mesh, sheet, patch, and sponge.

Pharmaceutical Compositions

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue-specific antibody. The liposomes will be targeted to and taken up selectively by the organ.

The present invention also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other apoptotic agents, including, but not limited to IL-10.

Administration of the compositions of this invention may be carried out, for example, by parenteral, by intravenous, intratumoral, subcutaneous, intramuscular, or intraperitoneal injection, or by infusion or by any other acceptable systemic method. Formulations for administration of the compositions include those suitable for rectal, nasal, oral, topical (including buccal and sublingual), vaginal or parenteral (including subcutaneous, intramuscular, intravenous and intradermal) administration. The formulations may conveniently be presented in unit dosage form, e.g. tablets and sustained release capsules, and may be prepared by any methods well known in the art of pharmacy.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension the composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

### Cells of the Invention

In one aspect, the invention includes isolating a stem cell from a subject. Therefore, the invention also provides methods of isolating, culturing and expansion of stem cells. In one embodiment the stem cells include, but are not limited, to BMPACs, bone marrow progenitors, endothelial progenitors, cardiac progenitor cells, mesenchymal stem cells as well as embryonic stem cells and induced pluripotent cells and their progenitor derivatives.

BMPACs of the invention and their progeny can be sterile, and maintained in a sterile environment. Such BMPAC, pluralities, populations, and cultures thereof can also be included in a medium, such as a liquid medium suitable for administration to a subject (e.g., a mammal such as a human).

Methods for isolating BMPACs are provided herein. In one embodiment stem cells are isolated from a subject by density-gradient centrifugation with Histopaque-1083. In another embodiment, the stem cells are separated from macrophages. Separation of stem cells from macrophages can be carried out through many methods known in the art including, but not limited to, contacting the mixture of macrophages and stem cells with an uncoated plate, wherein macrophages attach to the plate; collecting the unattached cells comprising the stem cells; and contacting the unattached cells with a on culture dish coated with 5 μg/ml human fibronectin.

In one embodiment, the stem cells are cultured in a culture medium. In some embodiments the culture medium comprises phenol red-free endothelial cell basal medium-2, fetal bovine serum, vascular endothelial growth factor-A, fibroblast growth factor-2, epidermal growth factor, insulin-like growth factor-1, ascorbic acid, and antibiotics.

In various embodiments, storing, stored, preserving and preserved stem cells and conditioned medium include freezing (frozen) or storing (stored) BMPACs and conditioned medium, such as, for example, individual BMPAC, a population or plurality of BMPACs, a culture of BMPACs, co-cultures and mixed populations of BMPACs and other cell types and conditioned medium. BMPACs and their conditioned medium can be preserved or frozen, for example, under a cryogenic condition, such as at −20° C. or less, e.g., −70° C. Preservation or storage under such conditions can include a membrane or cellular protectant, such as dimethylsulfoxide (DMSO).

BMPACs, a population or plurality or culture of BMPACs, progeny of BMPACs (e.g., any clonal progeny or any or all various developmental, maturation and differentiation stages) and conditioned medium of BMPACs can be can be administered to a subject, or used to implant or transplant as a cell-based or medium based therapy, or to provide factors, provide a benefit to a subject (e.g., by inhibiting miR-375 in BMPACs in the subject to treat ischemic injury).

### Methods of Treatment

The invention contemplates use of the cells of the invention in both in vitro and in vivo settings. Thus, the invention provides for use of the cells of the invention for research purposes and for therapeutic or medical/veterinary purposes. In research settings, an enormous number of practical applications exist for the technology.

In accordance with the invention, methods of providing a cellular therapy and methods of treating a subject having a disease or disorder that would benefit from a cellular therapy are provided. In one embodiment, a method includes administering at least one cell in which miR-375 has been inhibited in an amount sufficient to provide a benefit to the subject. In some embodiments, the subject having a disease or disorder has ischemic injury, such as heart failure ischemic injury. BMPACs, the progeny of BMPACs, or conditioned medium of BMPACs or their progeny can be administered or delivered to a subject by any route suitable for the treatment method or protocol. Specific non-limiting examples of administration and delivery routes include parenteral, e.g., intravenous, intramuscular, intrathecal (intra-spinal), intrarterial, intradermal, subcutaneous, intra-pleural, transdermal (topical), transmucosal, intra-cranial, intra-ocular, mucosal, implantation and transplantation.

In some embodiments, the BMPACs or their progeny can be autologous with respect to the subject; that is, the BMPACs used in the method (or to produce the conditioned medium) were obtained or derived from a cell from the subject that is treated according to the method. In other embodiments, the BMPACs, the progeny of BMPACs or conditioned medium of BMPACs or their progeny can be allogeneic with respect to the subject; that is, the BMPACs used in the method (or to produce the conditioned medium) were obtained or derived from a cell from a subject that is different from the subject that is treated according to the method.

The methods of the invention also include administering BMPACs, progeny of BMPACs, or conditioned medium of BMPACs prior to, concurrently with, or following administration of additional pharmaceutical agents or biologics. Pharmaceutical agents or biologics may activate or stimulate BMPACs or their progeny. Non-limiting examples of such agents include, for example, IL-10.

In one embodiment, the cell is genetically modified in vivo in the subject in whom the therapy is intended. In certain aspects, for in vivo, delivery the nucleic acid is injected directly into the subject. For example, in one embodiment, the nucleic acid is delivered at the site where the composition is required. In vivo nucleic acid transfer techniques include, but is not limited to, transfection with viral vectors such as adenovirus, Herpes simplex I virus, adeno-associated virus), lipid-based systems (useful lipids for lipid-mediated transfer of the gene are DOTMA, DOPE and DC-Chol, for example), naked DNA, and transposon-based expression systems. Exemplary gene therapy protocols see Anderson et al., Science 256:808-813 (1992). See also WO 93/25673 and the references cited therein. In certain embodiments, the method comprises administering of RNA, for example mRNA, directly into the subject (see for example, Zangi et al., 2013 Nature Biotechnology, 31: 898-907).

For ex vivo treatment, an isolated cell is modified in an ex vivo or in vitro environment. In one embodiment, the cell is autologous to a subject to whom the therapy is intended. Alternatively, the cell can be allogeneic, syngeneic, or xenogeneic with respect to the subject. The modified cells may then be administered to the subject directly.

One skilled in the art recognizes that different methods of delivery may be utilized to administer an isolated nucleic acid into a cell. Examples include: (1) methods utilizing physical means, such as electroporation (electricity), a gene gun (physical force) or applying large volumes of a liquid (pressure); and (2) methods wherein the nucleic acid or vector is complexed to another entity, such as a liposome, aggregated protein or transporter molecule.

The amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

In methods of treatment, a method may be practiced one or more times (e.g., 1-10, 1-5 or 1-3 times) per day, week, month, or year. The skilled artisan will know when it is appropriate to delay or discontinue administration. Frequency of administration is guided by clinical need or surrogate markers. An exemplary non-limiting dosage schedule is every second day for a total of 4 injections, 1-7 times per week, for 1, 2, 3, 4, 5, 6, 7, 8, 9, 10, 15, or more weeks, and any numerical value or range or value within such ranges.

Of course, as is typical for any treatment or therapy, different subjects will exhibit different responses to treatment and some may not respond or respond less than desired to a particular treatment protocol, regimen or process. Amounts effective or sufficient will therefore depend at least in part upon the disorder treated (e.g., the type or severity of the disease, disorder, illness, or pathology), the therapeutic effect desired, as well as the individual subject (e.g., the bioavailability within the subject, gender, age, etc.) and the subject's response to the treatment based upon genetic and epigenetic variability (e.g., pharmacogenomics).

In some aspects, the invention provides a method of ex vivo treatment, wherein the method comprises isolating a stem cell from a subject; inhibiting miR-375 in the stem cell; and administering the stem cell to the subject. In some embodiments, the method further comprises activating PDK-1 in the stem cell. In another embodiment, the method further comprises administering an effective amount of IL-10 to the subject. In certain embodiments, the method treats ischemic injury.

In one aspect, the invention provides a method of improving cardiac function in a subject with myocardial infarction (MI). In one embodiment, the method comprises inhibiting miR-375 in a BMPAC to produce a modified BMPAC and administering therapeutically effective amount of the modified BMPAC. In another embodiment, the method comprises administering therapeutically effective amount of an inhibitor of miR-375. In yet another embodiment, the cardiac function is measured by ejection fraction (EF) or fractional shortening (ES). In one embodiment, the improvement of cardiac function comprises an improvement in EF or ES.

In another aspect, the invention provides a method of in vivo treatment, wherein the method comprises administering an effective amount of an inhibitor of miR-375. In one embodiment, the method further comprises administering an effective amount of a PDK-1 activator. In other embodiments, the method further comprises administering an effective amount of IL-10.

In some embodiments, the inhibitor of miR-375 includes, but is not limited to, a nucleic acid, a small molecule, a peptide and an antibody. In some embodiments, the PDK-1 activator includes, but is not limited to, a nucleic acid, a small molecule, a peptide and an antibody.

### Kits

The present invention also pertains to kits useful in the methods of the invention. Such kits comprise various combinations of components useful in any of the methods described elsewhere herein, including for example, hybridization probes or primers (e.g., labeled probes or primers), antibodies, reagents for detection of labeled molecules, materials for the amplification of nucleic acids, medium, media supplements, components for deriving an insulin-producing cell derived from an BMPACs, an BMPACs cell, and instructional material. For example, in one embodiment, the kit comprises components useful for deriving a BMPAC and inhibiting miR-375 is the derived cell

The invention further provides kits, including BMAPCs, populations or a plurality of BMAPCs, cultures of BMAPCs, co-cultures and mixed populations of BMAPCs, progeny differentiated BMAPCs of any developmental, maturation or differentiation stage, as well as conditioned medium produced by contact with BMAPCs or their progeny, packaged into suitable packaging material. In various non-limiting embodiments, a kit includes an insulin-producing cell derived from a BMAPCs. In various aspects, a kit includes instructions for using the kit components e.g., instructions for performing a method of the invention, such as culturing, expanding (increasing cell numbers), proliferating, differentiating, maintaining, or preserving BMAPCs or their progeny, or a cell based treatment or therapy. In various aspects, a kit includes an article of manufacture, for example, an article of manufacture for culturing, expanding (increasing cell numbers), proliferating, differentiating, maintaining, or preserving BMAPCs or their progeny, such as a tissue culture dish or plate (e.g., a single or multi-well dish or plate such as an 8, 16, 32, 64, 96, 384 and 1536 multi-well plate or dish), tube, flask, bag, syringe, bottle or jar. In additional various aspects, a kit includes an article of manufacture, for example, an article of manufacture for administering, introducing, transplanting, or implanting pluripotent stem cells into a subject locally, regionally or systemically.

A label or packaging insert can include appropriate written instructions, for example, practicing a method of the invention. Thus, in additional embodiments, a kit includes a label or packaging insert including instructions for practicing a method of the invention in solution, in vitro, in vivo, or ex vivo. Instructions can therefore include instructions for practicing any of the methods of the invention described herein. Instructions may further include indications of a satisfactory clinical endpoint or any adverse symptoms or complications that may occur, storage information, expiration date, or any information required by regulatory agencies such as the Food and Drug Administration for use in a human subject.

The instructions may be on “printed matter,” e.g., on paper or cardboard within the kit, on a label affixed to the kit or packaging material, or attached to a tissue culture dish, tube, flask, roller bottle, plate (e.g., a single multi-well plate or dish such as an 8, 16, 32, 64, 96, 384 and 1536 multi-well plate or dish) or vial containing a component (e.g., pluripotent stem cells) of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

The kits of the invention can additionally include growth medium, buffering agent, a preservative, or a cell stabilizing agent. Each component of the kit can be enclosed within an individual container or in a mixture and all of the various containers can be within single or multiple packages.

BMAPCs or their progeny, as well as conditioned medium produced by contact with BMAPCs or their progeny, can be packaged in dosage unit form for administration and uniformity of dosage. “Dosage unit form” as used herein refers to physically discrete units suited as unitary dosages; each unit contains a quantity of the composition in association with a desired effect. The unit dosage forms will depend on a variety of factors including, but not necessarily limited to, the particular composition employed, the effect to be achieved, and the pharmacodynamics and pharmacogenomics of the subject to be treated.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Negative Regulation of miR-375 by Interleukin-10 Enhances Bone Marrow-Derived Progenitor Cell-Mediated Myocardial Repair and Function after Myocardial Infarction

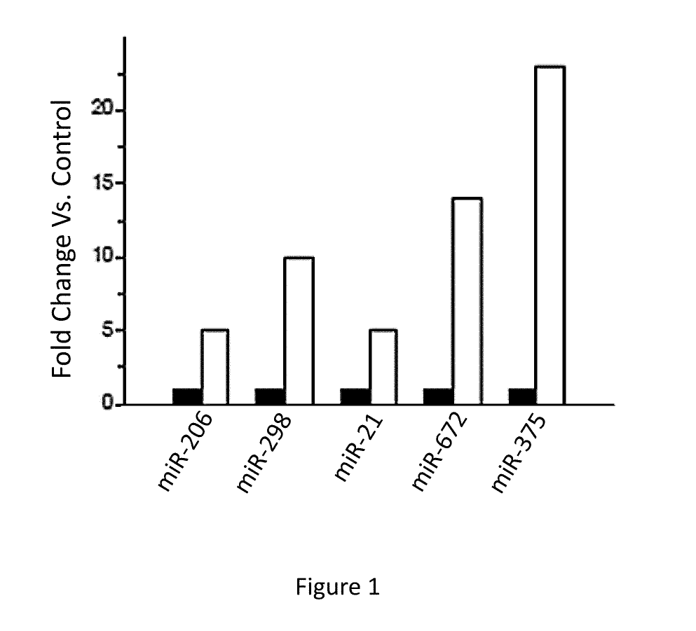

The results presented herein demonstrate the first evidence of miR-375 to be an independent downstream target of IL-10 and miR-375 knockdown in one marrow-derived angiogenic progenitor cells (BMPACs) improve their retention and function in ischemic myocardium. It is herein shown that miR-375 expression is significantly up regulated in BMPACs upon exposure to either inflammatory or hypoxic stimulus (FIG. 1) and that inhibition of IL-10 dependent miR-375 exhibits pleiotropic beneficial effects, which contribute to the enhanced BMPAC survival and angiogenic potential.

This data identifies a novel signaling that determines the survival and tissue repair function of adoptively transferred stem cells in ischemic myocardium. Moreover, these findings have a clear translational value. Bone marrow derived progenitor cells have been used in clinical trials of heart failure with only modest success rate. One of the recognized limitations of stem cell based therapies is extremely low retention, survival and alteration in the function of transplanted stem cells in the ischemic and inflamed myocardium, thereby compromising the full functional benefits of cell based therapies. Evidence is provided herein that ex vivo modulation of miR375 expression in EPCs before transplantation not only enhances survival but also repair function of these cells. Finally, this is the first demonstration of a negative role of miR375 in cardiovascular injury context and potentially be applicable to other types of stem cell therapies for ischemic tissue repair.

The materials and methods employed in these experiments are now described.

Bone Marrow Cell Isolation and BMPAC Culture

BMPAC isolation, ex vivo expansion and culture of BMPACs was performed as described previously (Krishnamurthy et al., 2009, Circ Res 104:e9-18). The BMPACs are phenotypically akin to mouse bone marrow-derived endothelial progenitor cells and have widely published. Given the ambiguity over exact definition of mouse EPCs, these cells are referred to as BMPACs herein. In brief, bone marrow mononuclear cells were isolated from mice by density-gradient centrifugation with Histopaque-1083 (Sigma-Aldrich, St. Louis, Mo.) and macrophage-depleted by allowing attachment to uncoated plate for 1 hour. The unattached cells were removed and plated on culture dishes coated with 5 μg/ml human fibronectin (Sigma) and cultured in phenol red-free endothelial cell basal medium-2 (EBM-2, Clonetics) supplemented with 5% fetal bovine serum (FBS), vascular endothelial growth factor (VEGF)-A, fibroblast growth factor-2, epidermal growth factor, insulin-like growth factor-1, ascorbic acid, and antibiotics (All from Lonza, Clonetics, Walkersville, Md.). Cells were cultured at 37° C. with 5% CO₂ in a humidified atmosphere. After 4 days in culture, non-adherent cells were removed by washing with phosphate-buffered saline, new medium was applied, and the culture was maintained through day 7. BMPACs, recognized as attaching spindle-shaped cells were used for further analysis and treatment. In all the experiments wherever mentioned, BMPAC were lipofectamine mediated transfected with miRNA inhibitor or mimic or respective controls (30 nM) for 24 hours. For lentiviral infection, BMPACs were transduced with lentivirus-green fluorescent protein (GFP) (3.2×10⁵ PFU/ml) for 24 or 48 hours.

LPS or Hypoxia-Induced miR-375 Expression in BMPAC (In Vitro Studies)

BMPACs derived from bone marrow of WT (WT-BMPAC) and IL-10 KO mice (IL-10-deficient; KO-BMPAC) were subjected to LPS (100 ng/ml, Sigma-Aldrich, St. Louis, Mo.) insult or incubated in 1% 02 for 24 hours and treated with or without IL-10 (10 ng/ml, R&D systems). WT-BMPACs were treated with LPS or IL-10 or both before the addition of 5 μg actinomycin D (Act-D, Sigma-Aldrich, St. Louis, Mo.). At increasing intervals (0, 30, 60, and 120 minutes) thereafter, cells were processed and changes in the amount of miR-375 were quantified by quantitative real-time polymerase chain reaction (RT-PCR).

Tube Formation Assay

WT-BMPAC and IL-10 KO-BMPAC were treated with scrambled control (ctrl) or anti-miR-375 for 24 hours and media supernatant from the same was collected and added in equal quantities (1:1) of growth factor reduced EBM-2 media with 10% FBS (All from Lonza, Clonetics, Walkersville, Md.) to 1.5×10⁴ human umbilical vein endothelial cells (HUVECs) plated on 40 μl Matrigel (BD Falcon) in a 96-well plate. After incubation at 37° C. in an atmosphere of 5% CO₂ gels were observed using a phase contrast microscope (×4). The branch points for each tube structure were counted in each image. Results are represented as SEM for three independent experiments.

Apoptosis Assay

WT-BMPAC and IL-10 KO-BMPAC were treated with scrambled or

Anti miR-375 for 24 hours. Thereafter, cells were subjected hydrogen peroxide (H₂O₂) insult (100 μm) for 2 hours and cells were evaluated for apoptosis by terminal deoxynucleotidyl transferase-mediated dUTP nick end-labeling (TUNEL) staining and activity of caspase-3/caspase-7. Apoptosis was measured with the cell death detection kit (Cell death detection assay, Roche, Indianapolis, Ind.) following the manufacturer's instructions. The activity of caspase-3 and caspase-7 was detected in 96-well format using the caspase-Glo 3/7assay kit (Promega, Madison, Wis.) following the manufacturer's instructions. Results are presented as SEM for three independent experiments.

Proliferation Assay

WT-BMPAC and IL-10 KO-BMPAC were treated with scrambled or anti-miR-375 for 24 hours. Thereafter, CyQuant and 3-[4,5-dimethylthiazol-2-yl]-2,5 diphenyl tetrazolium bromide (MTT) assay were performed in 96-well plates (Corning) with a cell seeding of 1×10⁴ cells per 96 well, followed by incubation with CyQuant (Invitrogen, Carlsbad, Calif.) or MTT reagent (Sigma, St. Louis, Mo.) following the manufacturer instructions. Results are presented as SEM for the three independent experiments.

Real-Time Polymerase Chain Reaction

Expression levels of miR-375 were measured using quantitative miRNA stem loop RT-PCR technology (TaqMan miRNA assays; Applied Biosystems). This assay uses gene specific stem cell loop RT primers and TaqMan probes to detect mRNA or mature miRNA transcripts. Transcription was performed using 2 μg or 10 ng total RNA and the TaqMan miRNA RT kit (Applied Biosystems, Foster City, Calif., USA). Real-Time PCR was performed on an applied biosystems 770 apparatus using the TaqMan Universal PCR Master Mix, No AmpErase UNG (Applied Biosystems). The amplification steps consisted of initial denaturation at 95° C., followed by 40 cycles of denaturation at 95° C. for 15 seconds and annealing at 60° C. for 1 minute. The TaqMan specific primer 18S or U6 small nucleolar RNA was used for normalization with the threshold delta-delta cycle method (Gene Expression Macro; Bio-Rad, Hercules, Calif.).

Luciferase Assay

BMPACs were cultured (Standard BMPAC media). Further BMPACs were cotransfected with miRNA mimic mma-miR-375 or Anti miR-375 or corresponding controls (30 nM) (Applied Biosystems) and a reporter plasmid containing the 3′ UTR of phosphoinositide-dependent protein kinase-1 (PDK-1) inserted downstream of the luciferase reporter gene (pEZX-PDK-1-UTR; GeneCopoeia; Rockville, Md.) using Lipofectamine 2000 (Invitrogen) in a 48-well plate. Twenty-four hours after transfection, luciferase assay was performed on cell-culture supernatant using Secrete-Pair dual luminescence kit (GeneCopoeia; LabOmics, Rockville, Md.).

siRNA Experiments

Small interfering RNA (siRNA) sequences targeting mouse PDK-1 were synthesized by PDK-1 siRNA (Invitrogen, Carlsbad, Calif.) or a negative control (siRNA-NC) was used at a final concentration of 100 nm according to the manufacturer's instructions, and the cells were transfected for 24 hours. Subsequently, the knockdown efficiency in HUVECs was determined by Western blot assays. In addition, 30 nm anti-miR-375 (Applied Biosystems, Foster City, Calif., USA) was introduced alone or in combination with 100 nm PDK-1-1 SiRNA using lipofectamine RNAiMAX (Invitrogen, Carlsbad, Calif.) in HUVECs. The tube formation assay and apoptosis assay was then performed as described above.

MI and Study Design

Mice were subjected to infarction (MI) by ligation of left anterior descending (LAD) coronary artery as described previously (Krishnamurthy et al., 2010, FASEB J 24:2484-94; Krishnamurthy et al., 2009, Circ Res 104:e9-18). Immediately after LAD ligation, one set of mice received intramyocardial injection of 1×10⁵ GFP+WT-BMPAC (n=22) or IL-10 KO-BMPAC (n=12) BMPAC with or without miR-375 knockdown in a total volume of 15 μl at five different sites (basal anterior, mid anterior, mid lateral, apical anterior, and apical lateral) in the peri infarct area. All the mice were followed up for LV functional changes on 7, 14, and 28 days and structural remodeling at 28 days post-MI.

Echocardiography

Transthoracic two-dimensional M-mode echocardiogram was obtained using Vevo 770 (Visual Sonics, Toronto, Canada) equipped with 30 MHz transducer. Echocardiographic studies were performed before (baseline) and at 7, 14, and 28 day's post-MI on mice anesthetized with a mixture of 1.5% isoflurane and oxygen (1 l/min). M-mode tracings were used to measure. Percent fractional shortening (% FS) and percent ejection fraction (% EF) was calculated as described (Krishnamurthy et al., 2010, FASEB J 24:2484-94; Krishnamurthy et al., 2009, Circ Res 104:e9-18).

Morphometric Studies

The hearts were perfusion fixed with 10% buffered formalin. Hearts cut into three slices (apex, mid-LV, and base) and paraffin embedded. The morphometric analysis including infarct size and wall thickness and percent fibrosis was performed on Masson's trichrome stained tissue sections using Image-J software (NIH, Bethesda, version 1.30). Fibrosis area was measured to determine percent fibrosis (Krishnamurthy et al., 2009, Circ Res 104:e9-18).

Immunofluorescence for BMPAC Retention and Engraftment in Myocardial Tissue

Immunofluorescence staining for tissue sections was performed as described previously (Krishnamurthy et al., 2009, Circ Res 104:e9-18). Proliferation of BMPAC was assessed by green fluorescent protein (GFP)/5-bromo-2′-deoxyuridine (BrdU)+ cells in the border zone of infarcted myocardium and expressed as number per HVF at 5 days. Whereas for the mice received anti miR-375 WT/IL-10 BMPAC therapy capillaries formation was (Lectin positive) assessed in 10 randomly selected low-power visual fields (LPF) 28 days post-MI. Nuclei were counter-stained with 4′,6-diamidino-2-phenylindole (DAPI, 1:10000, Sigma Aldrich, St Louis, Mo.), and sections were examined with a fluorescent microscope (Nikon, Japan).

TUNEL Staining for Apoptosis in the Myocardium

At 5 day's post-MI, myocardial apoptosis was determined by TUNEL staining on 4 μm thick paraffin-embedded sections as per manufacturer's instructions (Cell death detection assay, Roche, Indianapolis, Ind.). Also BMPAC's were GFP⁺. DAPI staining was used to count the total number of nuclei. Counting the number of GFP+/TUNEL+ cells per HVF assessed apoptosis of transplanted BMPACs at 5 days post-MI. Whereas mice received scrambled/miR-375 knockdown BMPAC, TUNEL assay was performed counting the number of a-sarcomeric actinin TUNEL+ cells per HVF assessed apoptosis of transplanted BMPACs at 5 days post-MI.

Isolation of Neonatal Rat Cardiomyocytes and Treatments

Isolation of neonatal rat ventricular myocytes and treatments NRCM were prepared by enzymatic digestion of hearts obtained from newborn (0- to 2-day old) Sprague-Dawley rat pups using percoll gradient centrifugation and plated on six-well cell culture grade plates (coated with collagen IV) at a density of 0.85×10⁶ cells per well in DMEM/M199 medium and maintained at 37° C. in humid air with 5% CO₂. Cells were treated with BMPAC control conditioned medium or anti-miR-375 conditioned medium and subjected to 100 μM H₂O₂ stress and TUNEL assay was performed as mentioned above.

Statistical Analyses

Data are presented as mean±SE between two groups; unpaired Student's t-test determined significance. For >2 groups, analysis of variance with Turkey post hoc was used. p<0.05, <0.01, and <0.001 were considered significant.

The results of the experiments are now described.

IL-10 Regulates miR-375 Expression in BMPAC

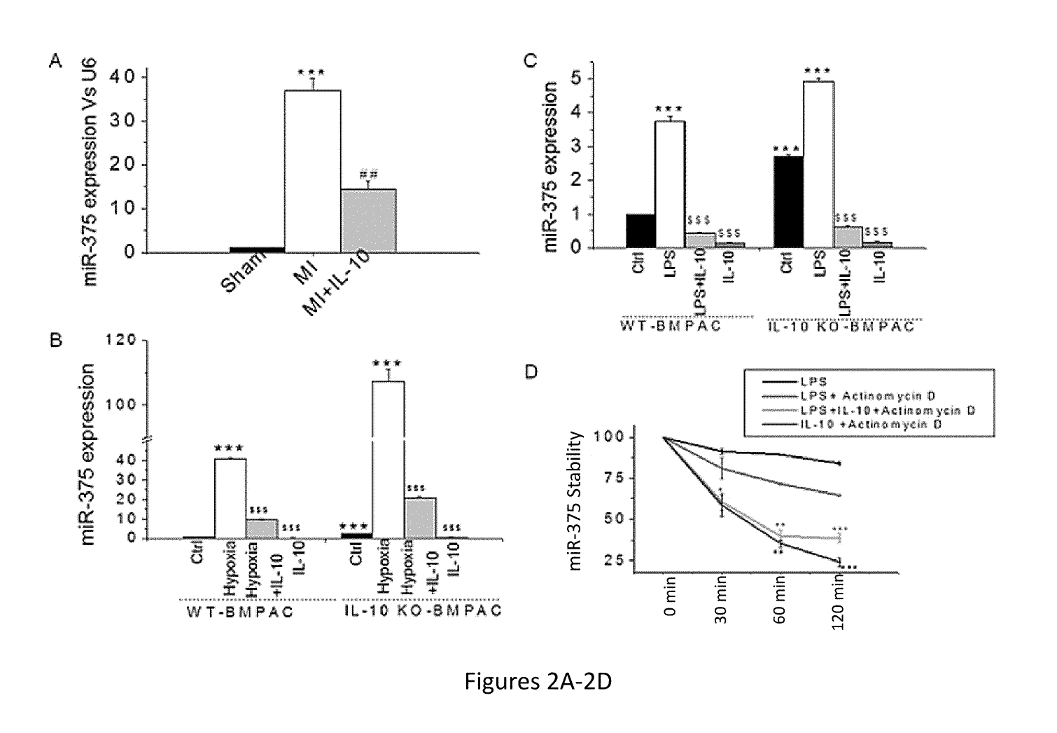

miR-375 levels in the LV tissue of the MI mice were found to be significantly higher compared with sham at 5 days post-MI (FIG. 2A). Exogenous recombinant IL-10 therapy substantially reduced miR-375 expression in the ischemic myocardium (FIG. 2A). As miR-375 has been identified to be robustly upregulated in mononuclear cells from IL-10 KO mice (Schaefer et al., 2011, J Immunol 187:5834-41) and the biological function of this miR has never been studied in cardiovascular physiology. miR-375 levels were measured after different stimulus in BMPAC. The LPS-dependent upregulation of miR-375 observed in BMPAC was significantly higher in BMPACs obtained from IL-10 KO animals, which also showed higher basal levels of miR-375 (FIG. 2B). Similarly compared with WT BMPAC, miR-375 was expressed at 15-fold higher level in IL-10 KO BMPACs cultured under hypoxic conditions (FIG. 2C) indicating that miR-375 is a stress marker for BMPAC and induced by inflammatory (LPS) and hypoxia and is in turn subject to negative regulation by IL-10. In the attempt to investigate the mechanism whereby IL-10 decreases miR-375 levels, the effect of IL-10 on miR-375 post-transcriptional stability was examined. BMPACs were stimulated with LPS with or without IL-10 and then were examined for the rate of miR-375 decay following blockade of further transcription using Act-D. At increasing intervals thereafter, cells were processed and changes in the amount of miR-375 were quantified by quantitative RT-PCR (qRT-PCR). The LPS-dependent miR-375 stability was observed in BMPAC. Interestingly IL-10 markedly reduces miR-375 half-life compared with LPS (FIG. 2D) these observations suggest that IL-10 controls miR-375 expression at post-transcriptional level and regulates inflammation and/or ischemia induced expression of miR 375.

Knockdown of miR-375 Enhances BMPAC Functions

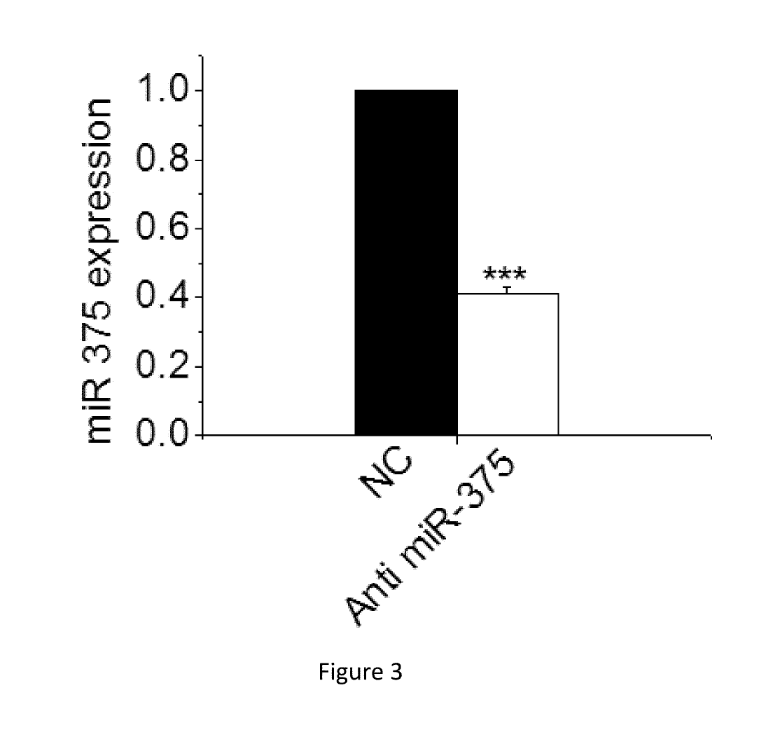

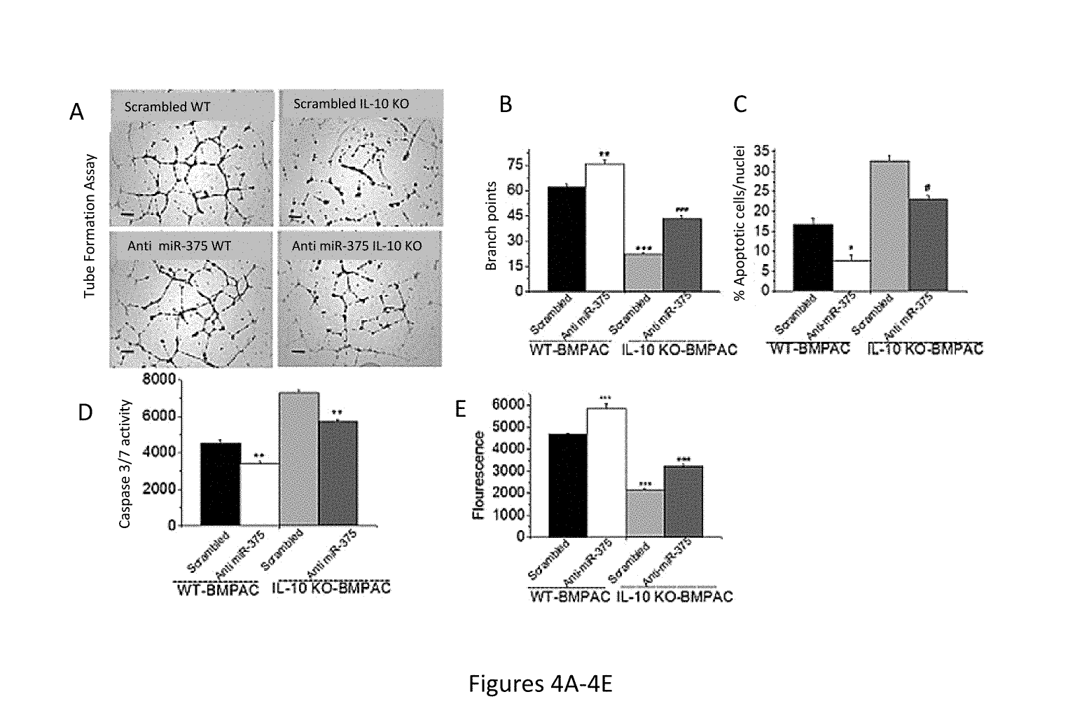

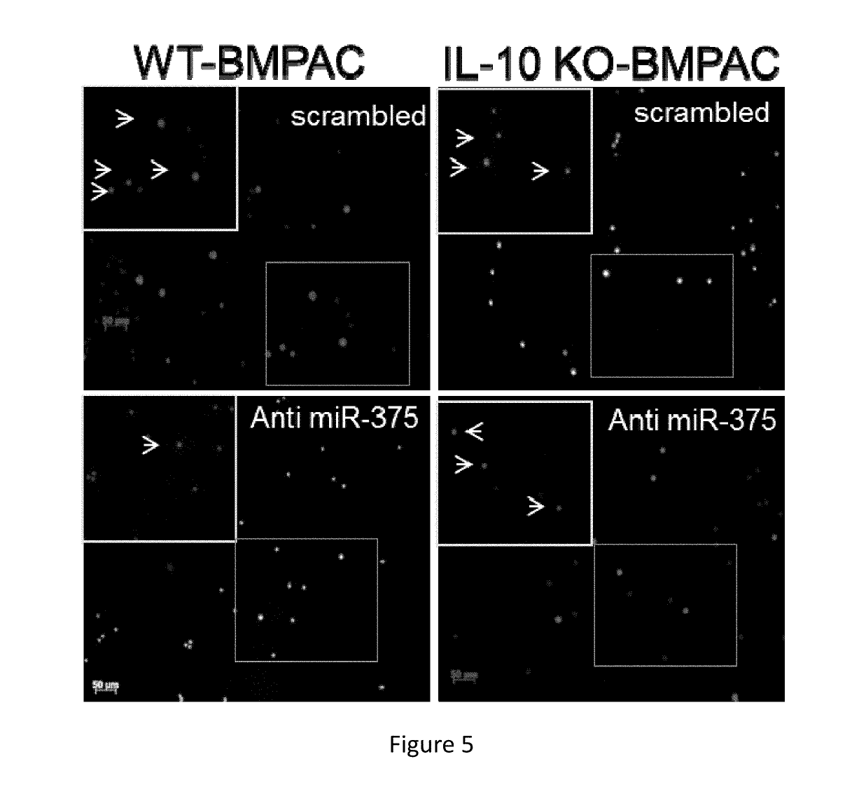

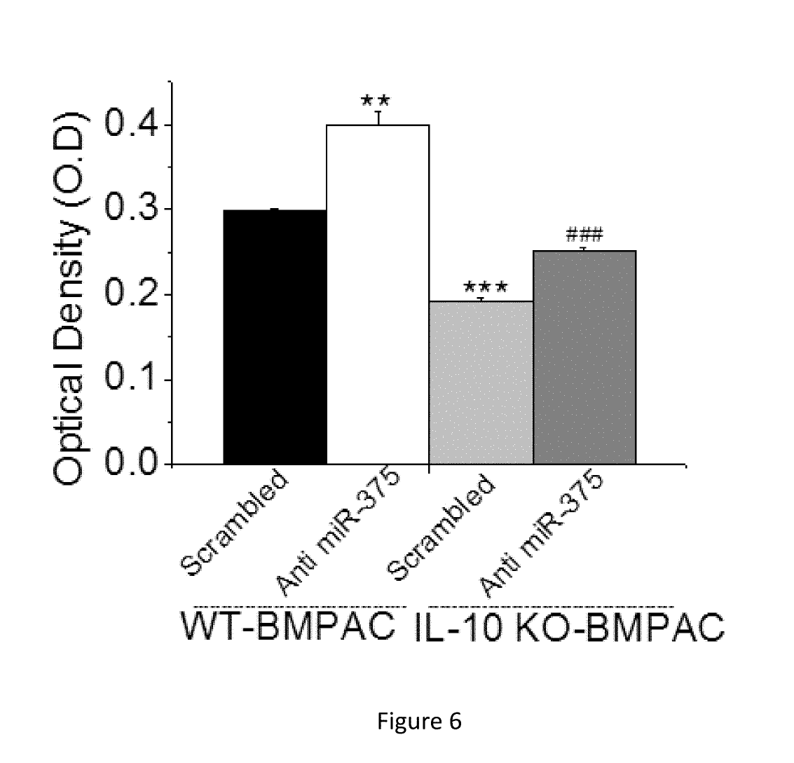

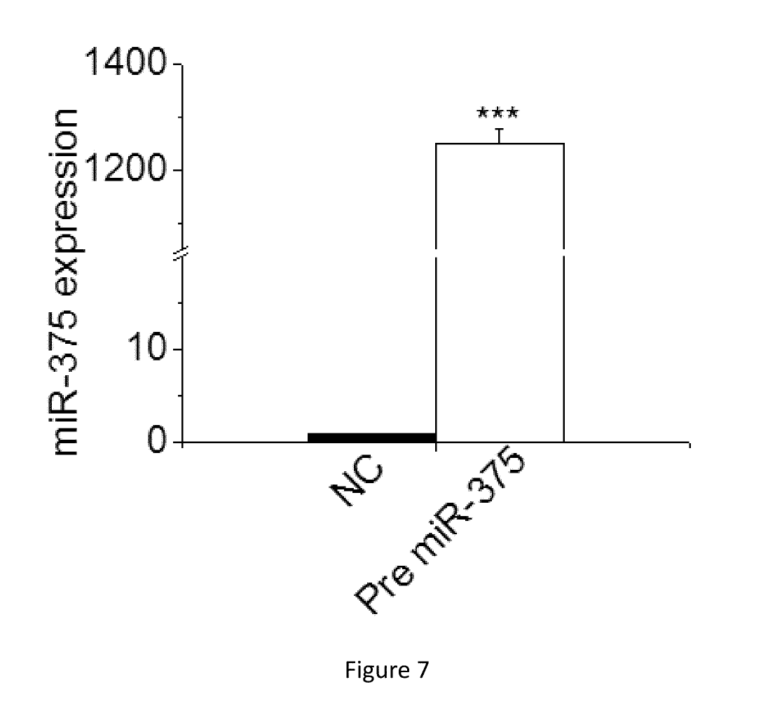

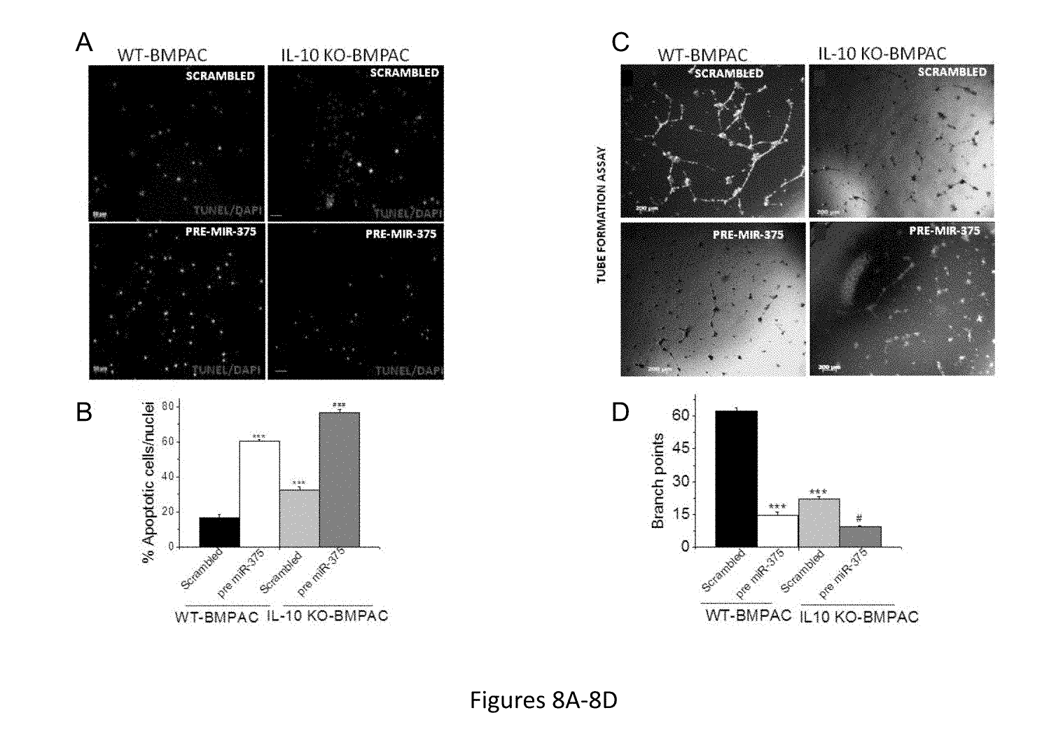

To enable the detailed study of the role of miR-375 biological function in BMPACs, BMPACs were transfected with anti-miR-375 or scrambled non-specific anti miRs. Transfection significantly repressed miR-375 as compared to scrambled BMPAC (FIG. 3). BMPACs functions further assessed were: tube formation, cell viability, and proliferation in both WT and IL-10 KO BMPACs. The exposure of BMPACs with anti-miR 375 significantly increased the tube formation ability compared with control cells. More interestingly, anti-miR-375 treatment to IL-10 KO BMPAC, which are functionally impaired and have poor tube formation ability, partially restored their tube formation ability suggesting IL-10 regulates miR-375-mediated BMPAC angiogenic activity (FIGS. 4A, 4B). Anti-miR-375 in WT-BMPAC significantly reduced apoptosis whereas miR-375 knockdown in IL-10 KO BMPAC partially reduced apoptosis exposed to H₂O₂ evident by both decreased TUNEL positive cells (FIG. 5) and quantification of TUNEL+ cells and caspase-3/-7 levels compared with their respective controls (FIGS. 4C, 4D), suggesting IL-10 regulates miR-375 mediated survival activity. Treatment of BMPACs with anti-miR-375 enhanced proliferation of WT-BMPAC compared to scramble BMPACs revealed by Ciquant assay (FIG. 4D) and MTT assay (FIG. 6). Whereas, anti-miR-375 treatment to IL-10 KO BMPAC partially restored proliferation compared with IL-10 KO scrambled BMPAC suggesting IL-10 regulates miR-375 mediated BMPAC proliferation. Interestingly miR-375 mimic treatment to both wild type and IL-10 KO BMPAC rendered them more prone to apoptosis and impaired tube formation ability on matrigel (FIGS. 7, 8). These observations suggest that IL-10 regulates miR-375-mediated BMPAC functions in vitro.

PDK-1 is a Direct Target of miR-375

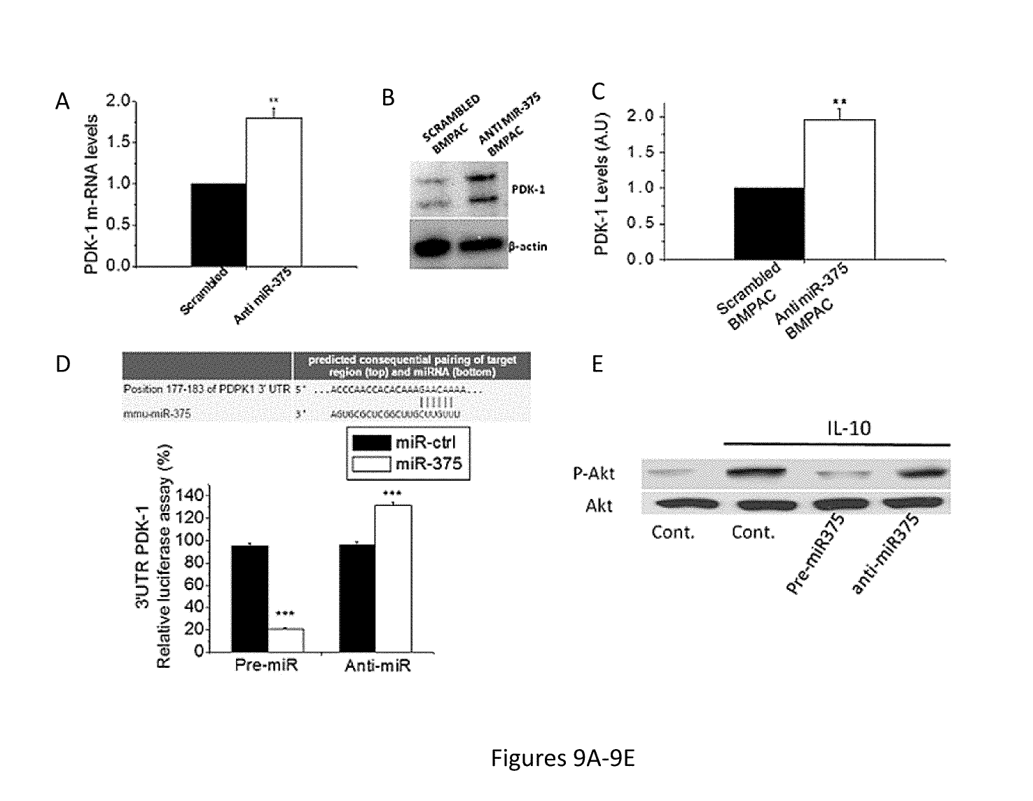

To identify potential targets of miR-375, Target Scan program designed to predict mRNA targets of miRs was used. One of the predicted targets for miR-375 is PDK-1. To investigate whether miR-375 affects PDK-1, anti-miR-375, or scrambled oligo were transfected in BMPAC. Subsequent analysis by real-time PCR (FIG. 9A) and western blot showed significant upregulation of PDK-1 in cells transfected with anti-miR-375, suggesting PDK-1 as a potential target for miR-375 in BMPAC (FIGS. 9B, 9C). To further confirm whether PDK-1 was a direct target of miR-375, luciferase assay was performed with the pEZX-PDK-1-UTR (vector with 3′-UTR of PDK-1) co-transfected into the BMPAC with miR-375 mimic or anti miR-375 or respective scrambled controls. Significantly decreased luciferase activity was observed with miR-375 mimic over expression as compared to scrambled negative control (FIG. 9D). Whereas anti miR-375 significantly increased luciferase activity (FIG. 9D) suggesting that miR-375 directly targets PDK-1. Since miR-375 is predicted to target PDK-1/AKT cell survival signaling, the effect of IL-10 on AKT-phosphorylation in BMPACs transfected with anti-miR-375 or pre-miR-375 was determined. In untransfected control BMPACs, IL-10 stimulated significant AKT-phosphorylation within 15 minutes; over-expression of miR-375 inhibited IL-10 responsiveness to AKT-phosphorylation while downregulation of miR-375 restored IL-10 sensitivity (FIG. 9E).

Effects of miR-375 on Angiogenesis and Apoptosis are PDK-1 Dependent

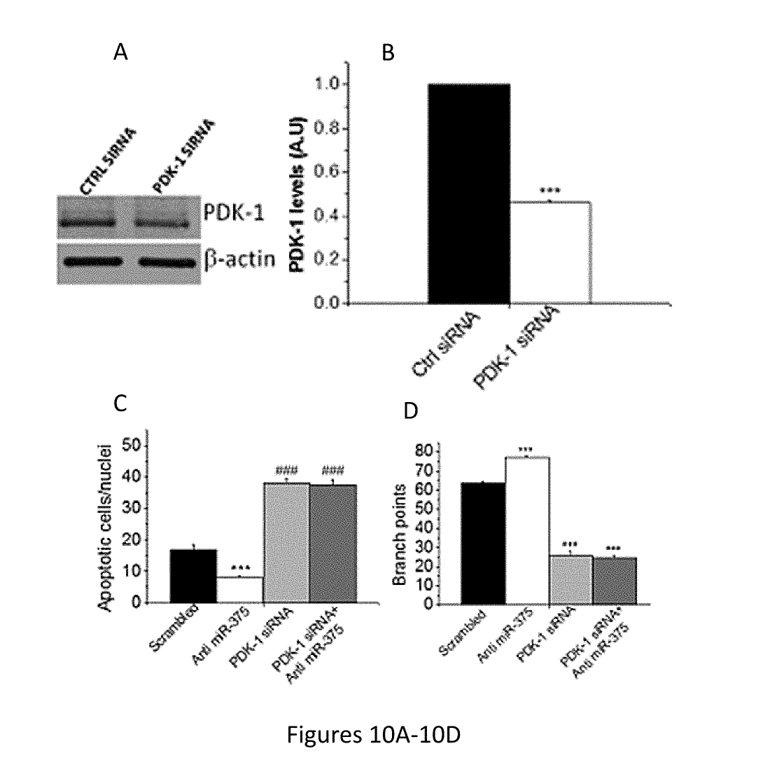

To investigate whether anti-miR-375 exerts its angiogenic and anti-apoptotic effects via PDK-1, PDK-1 was knocked down in human umbilical vein endothelial cells (HUVECs) using siRNA (FIGS. 10A, 10B) and further assessed their functions in apoptosis and tube formation assay. PDK-1 silencing exaggerated H₂O₂ induced apoptosis and inhibited tube formation compared with controls. Intriguingly in the co-transfection of PDK-1 siRNA and anti-miR-375 in HUVECs, the effect of anti-miR-375 on HUVECs apoptosis and tube formation ability was completely abolished by PDK-1 siRNA (FIGS. 10C, 10D). These data confirm that miR-375 enhances angiogenesis and reduces susceptibility to apoptosis in a PDK-1 dependent manner.

Increased Survival of miR-375 Knockdown BMPACs in the Heart Following MI

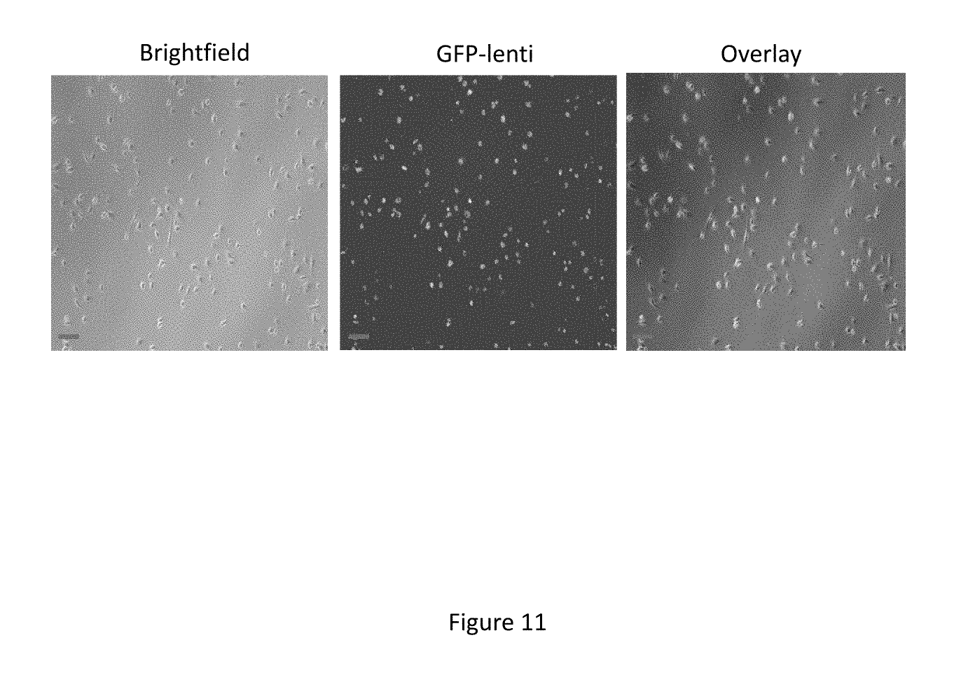

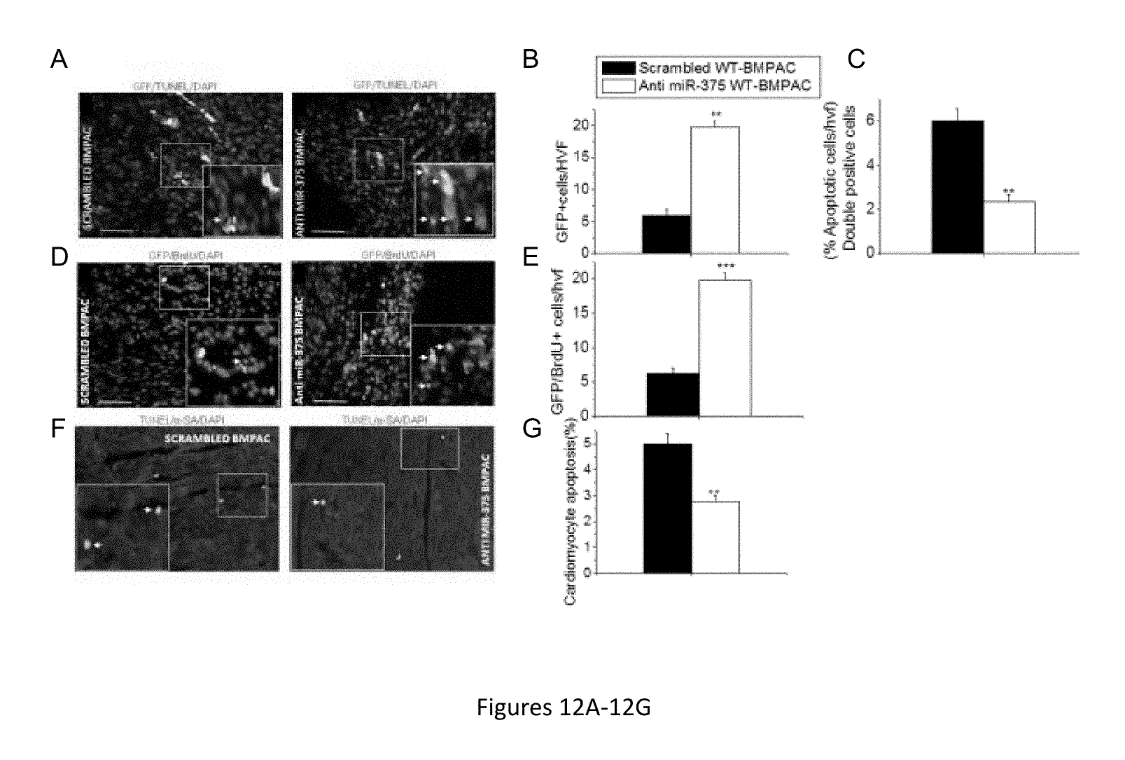

Superiority of miR-375 knockdown BMPACs in the face of H₂O₂ challenge relative to scrambled ctrl BMPACs in terms of survival, which was found in vitro, was further validated in vivo. BMPACs transduced with lentiviral GFP particles had >95% transduction efficiency (FIG. 11). The retention and survival of GFP+BMPACs was examined after their transplantation in the ischemic myocardium (5 days after MI). As shown in FIGS. 12A-12B, mice receiving miR-375 knockdown BMPACs had a higher number of GFP+BMPACs retained in the myocardium as compared to scrambled BMPAC ctrl. Interestingly, in scrambled BMPAC ctrl group, a large number of these GFP+ cells were undergoing apoptosis as compared to the mice that received miR-375 knockdown BMPACs (FIG. 12C). Furthermore, BMPACs engineered with miR-375 knockdown showed typical characteristics of increased proliferation observed in vitro was further validated in vivo. BrdU⁺/GFP⁺ cells were also significantly increased in miR-375 knockdown BMPACs compared with scrambled ctrl BMPACs indicating increased proliferation of the transplanted BMPACs (FIGS. 12D-12E). The cardiomyocyte apoptosis was also examined after anti-miR-375 treated BMPACs transplantation in the border zone of the infarct (5 days after MI). Interestingly, in scrambled BMPAC group, a large number of these cells were undergoing apoptosis as compared to the mice that received anti-miR 375 BMPAC (FIGS. 12F-12G). This data suggest that miR-375 knockdown BMPACs protects cardiomyocyte apoptosis in the ischemic myocardium. Collectively these data suggest that miR-375 knockdown in BMPACs protects transplanted BMPAC in ischemic myocardium and thereby increases the numeric availability (retention) of live BMPACs leading to enhanced myocardial repair and LV function.

PDK-1 is Upregulated in the Anti-miR-375 BMPACs Transplanted Hearts After MI

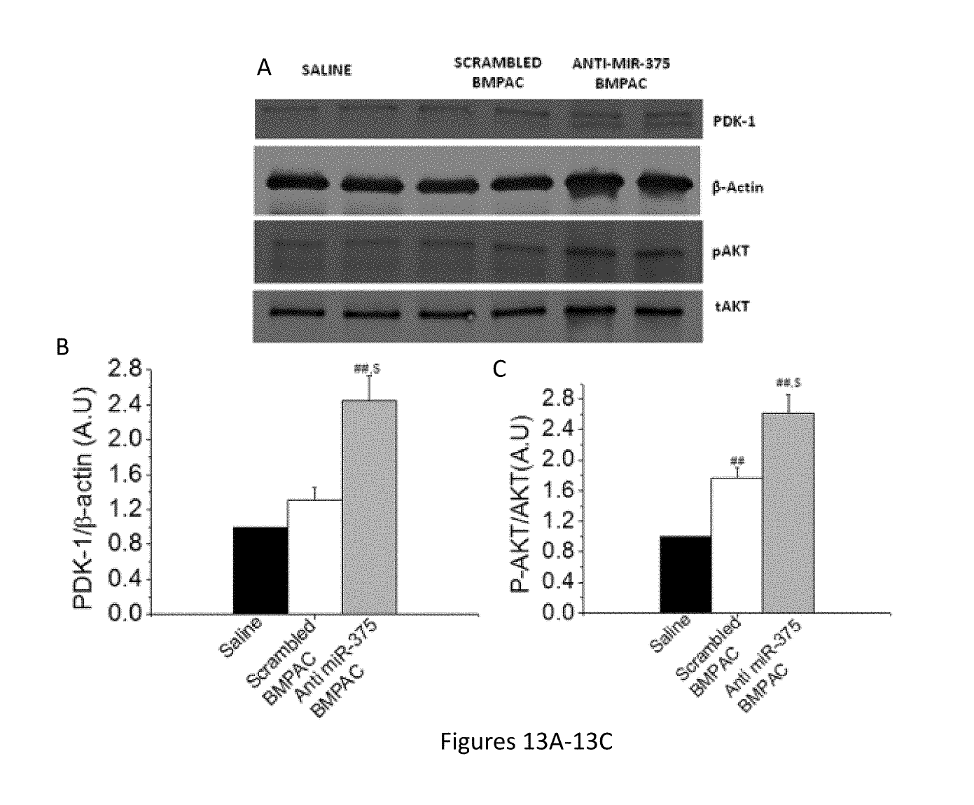

As PDK-1 is a potential target of miR-375 and also PDK-1 plays an important role in survival following MI (Ito et al., 2009, PNAS 106:8689-94; Mora et al., 2003, EMBO J 22:4666-76). Therefore, PDK-1 protein expression (FIG. 13) and its downstream target AKT in the border zone of infarct at 5 days post-MI were examined. Cardiomyocyte survival was associated with increased PDK-1 levels and AKT phosphorylation after MI. These data suggest that the miR-375-knockdown-mediated increase in PDK-1 expression was directly associated with the suppression of post-MI apoptosis.

Anti-miR-375 BMPACs Transplantation Attenuates Adverse LV Remodeling and Function after MI

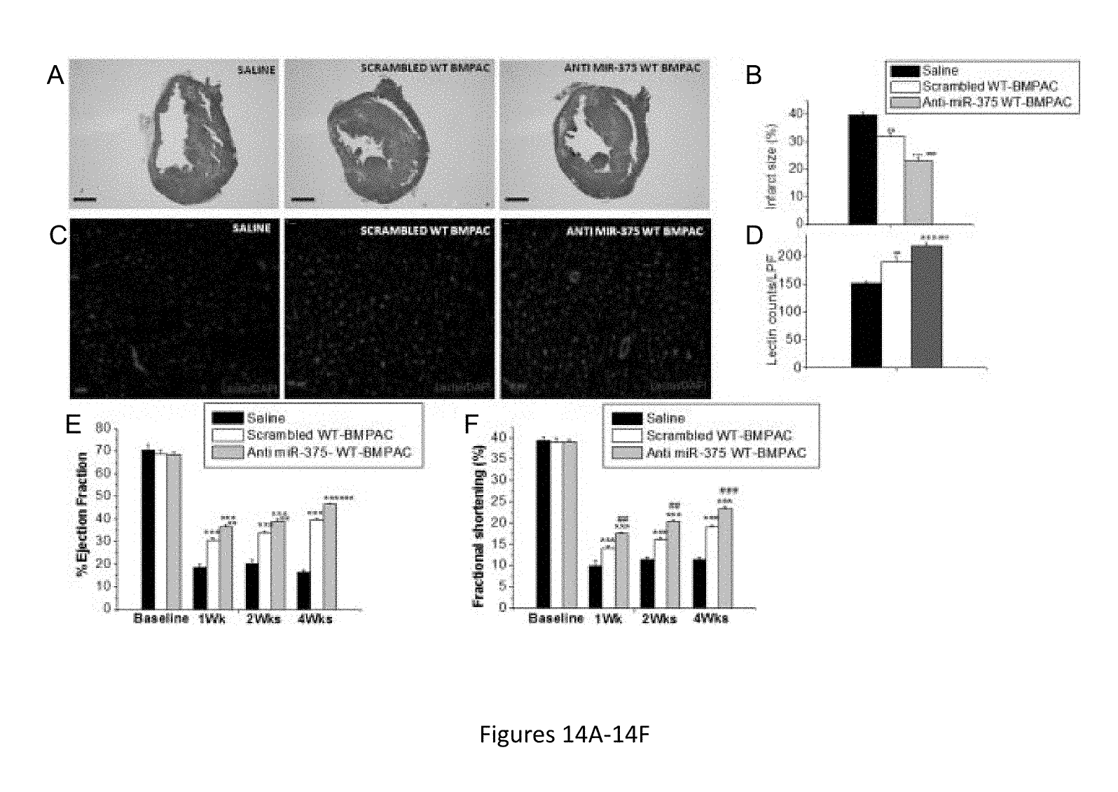

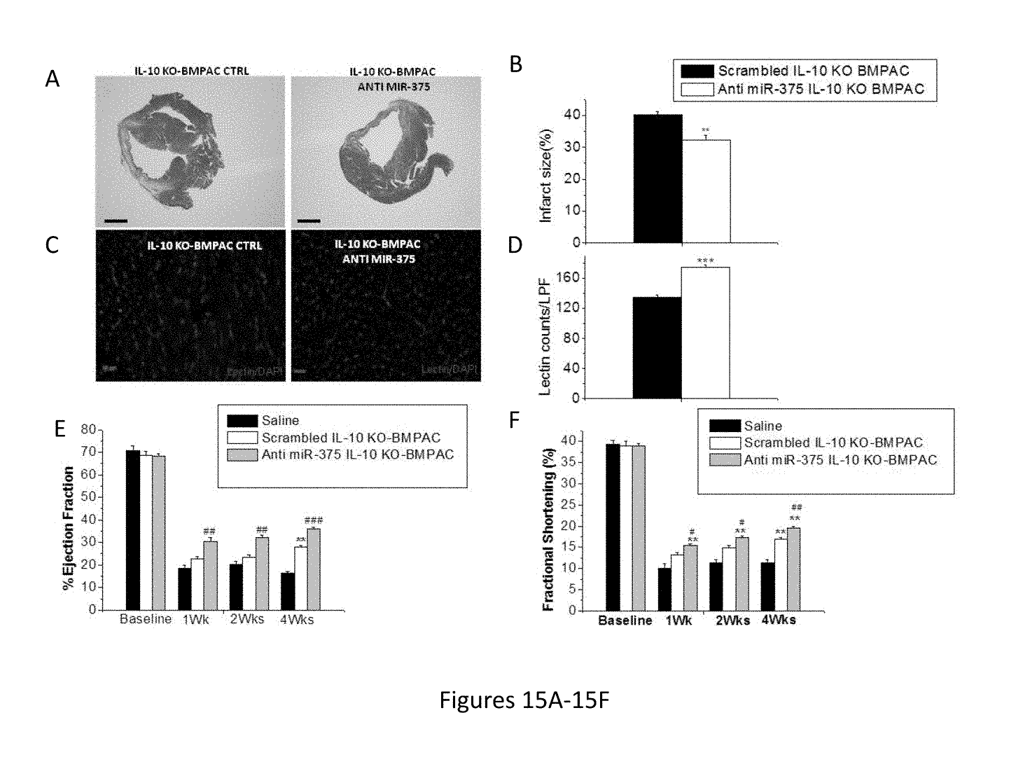

To assess the influence of anti-miR-375 on BMPAC-mediated effects on LV remodeling; % fibrosis area was assessed at 28 days after MI. Scrambled BMPAC significantly attenuated % fibrosis (FIGS. 14A-14B). Interestingly, anti-miRNA 375-BMPAC-treated mice showed further reduction in fibrosis (FIG. 14A-14B). Whereas IL-10 KO BMPAC treated with anti-miR-375 partially restored reduction in % fibrosis compared with scramble IL-10 KO BMPAC suggesting the role of IL-10 in regulating miR-375 in BMPAC mediated improvement in cardiac functions (FIG. 15).

To determine the effect of miR-375 knockdown on BMPAC-mediated neovascularization, capillary density was assessed in the border zone of the infarct. As shown in FIGS. 14C-14D, the number of Lectin+ capillaries were significantly higher in mice receiving anti-miR-375 treated BMPACs compared with those receiving scrambled miR-treated BMPACs. Interestingly IL-10 KO BMPAC treated with anti-miR-375 partially enhanced neovascularization compared with scramble IL-10 KO BMPAC suggesting the role of IL-10 in regulating miR-375 in BMPAC mediated improvement in cardiac functions (FIG. 15).

Since this data in the preceding experiments established that IL-10 suppresses miR-375 expression, it was determined whether ex vivo knockdown of miR-375 in BMPACs before transplantation mimics IL-10 protective effects at 7, 14, and 28 days, post-MI. As expected and in support of number of published studies mice that received scrambled miR-treated BMPAC (control) showed significantly increased % EF and % FS (FIGS. 14E, 14F) at 28 days, post-MI as compared to placebo (saline). Interestingly, anti-miR-375-treated BMPACs robustly enhanced increase in % EF and % FS (FIGS. 14I, 14J). Interestingly IL-10 KO BMPAC treated with anti-miR-375 partially restored cardiac functions compared with control IL-10 KO BMPACs (FIG. 15).

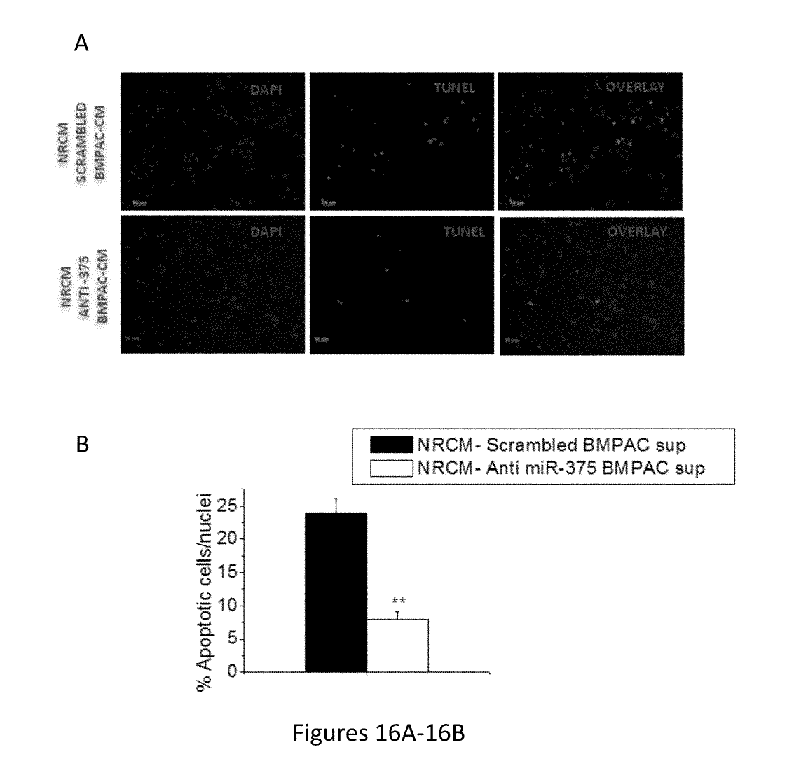

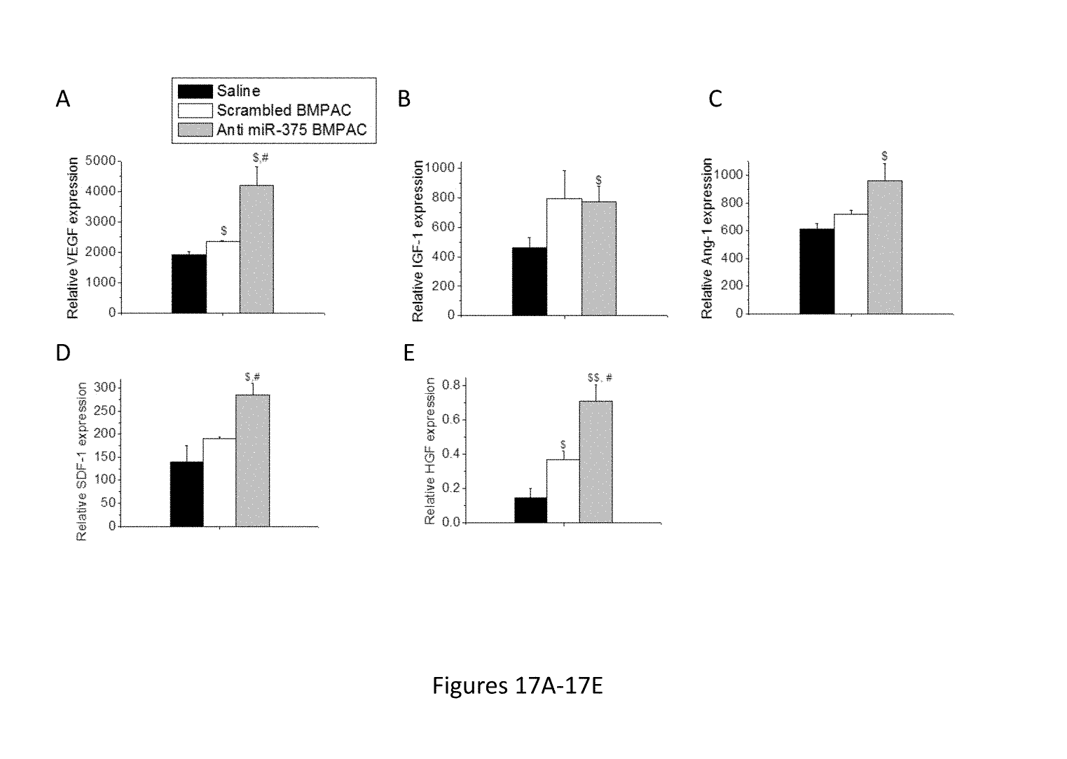

Interestingly, anti-miR-375 conditioned medium protected neonatal rat ventricular myocytes apoptosis (subjected to H₂O₂ injury) compared with scrambled ctrl BMPAC (FIGS. 16A, 16B). The paracrine activity of BMPAC was further validated in vivo and it was found that vascular endothelial growth factor (VEGF), hepatocyte growth factor (HGF), insulin growth factor-1 (IGF-1), angiopoetin-1 (Ang-1) and stromal derived factor-1 (SDF-1) were markedly increased in BMPAC treated with anti-miR-375 compared with scrambled ctrl and saline group in the myocardium at 28 days post-MI was assessed by quantitative RT-PCR (FIG. 17).

Inhibition of IL-10 Dependent miR-375 Exhibits Pleiotropic Beneficial Effects

Cellular therapy has emerged as a potential regenerative strategy for patients with AMI. In preclinical studies, BMPACs have been shown to enhance neovascularization and improve post-MI ventricular functions paving ways for BMPAC based clinical trials with modest success. However, the inflammatory and ischemic myocardial environment resulting in reduced survival and function of BMPAC/stem cells constitute important liabilities for autologous BMPAC/stem cell-based therapies, thereby compromising full benefits of post-infarct cardiovascular repair (Werner and Nickenig, 2006, Arterioscl Throm Vasc Biol 26:257-66; Ling et al., 2012, PLoS One 7:e50739). Combined therapy with bone marrow progenitor cells and IL-10 attenuates inflammatory response in the myocardium and also enhanced neovascularization and further improvement in LV function (Krishnamurthy et al., 2011, Circ Res 109:1280-9). Bone marrow progenitor cells from IL-10 KO mice display functional impairment and decreased survival when transplanted in ischemic myocardium (Krishnamurthy et al., 2011, Circ Res 109:1280-9).

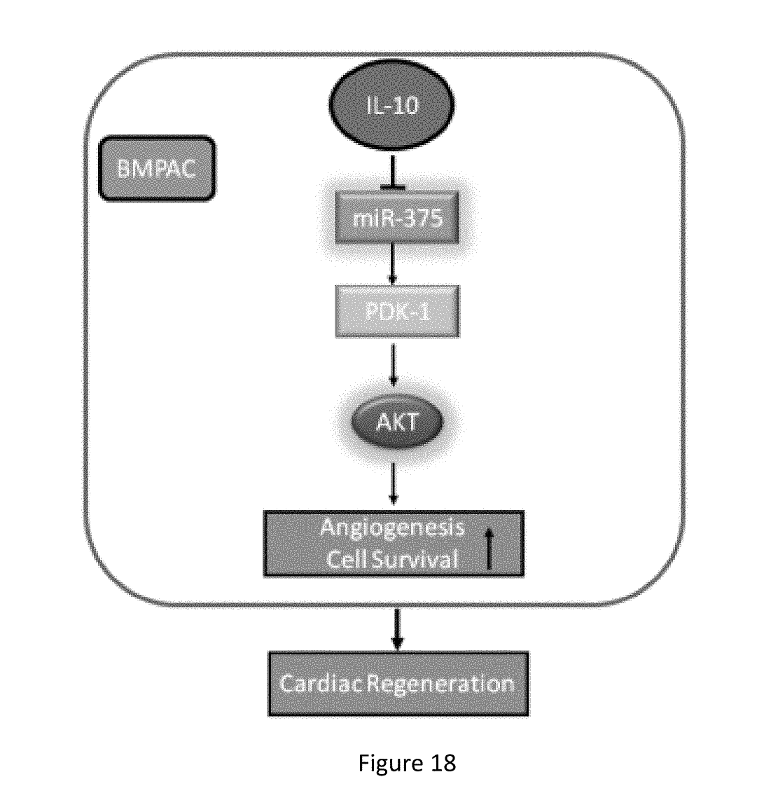

In this study, it was observed that IL-10 KO BMPACs show high basal levels of miR-375 and that exposure of wild type BMPAC to stress leads to upregulated miR-375 expression. Importantly, miR-375 was also observed to be elevated in LV tissue after MI and exogenous IL-10 therapy has an inhibitory effect on the same. As this has been an important miR related to cancers and since IL-10 deficient BMPAC show high levels of miR, this miR may play an important role in BMPAC-mediated angiogenesis and ischemic myocardium. Additionally, the biological function of this miR has never been studied in cardiovascular physiology although a single study reported elevated levels of miR-375 in HF patients (Akat et al., 2014, PNAS 111:11151-6). These results show that miR-375 indeed play a negative role both in the BMPAC survival and function as well in post-MI functional recovery by directly targeting PDK-1-Akt signaling pathways and IL-10 suppresses miR-375 expression. Several lines of evidence support these conclusions: (a) inflammation and hypoxia/ischemia up regulates miR-375 expression which is suppressed by IL-10; (b) Knockdown of miR-375 in BMPAC enhances their survival and functions functional rescue of IL-10 KO BMPAC, in vitro; (c) knockdown of miR-375 enhances the retention, survival and function of the intramyocardially transplanted BMPACs; (d) enhanced BMPAC survival in the ischemic myocardium is associated with augmentation of BMPAC-mediated neovascularization and further improvements in the LV function when compared with BMPAC transplantation alone (e) mechanistically, miR-375 effects on BMPACs appear to be mediated through PDK-1/AKT signaling mechanisms. On a whole, this study represents a logical extension of further mechanisms of IL-10 and is entirely novel since it is not focused upon IL-10 per se but on miR-375 as an independent downstream target of IL-10 activating PDK-1/AKT axis as illustrated in FIG. 18.

Prolonged inflammation has been implicated with reduced BMPAC mobilization, cell death, and functional impairment (Andreou et al., 2006, Atherosclerosis 189:247-54; Liu et al., 2012, Am J Med Sci 344:220-6). Interestingly, Schaefer et al. that reported that IL-10 KO mononuclear cells express high levels of miR-375 (Schaefer et al., 2011, J Immunol 187:5834-41), however whether miR-375 modulates BMPAC survival and function when subjected to inflammatory or hypoxia and the role of this miR in cardiovascular injury and repair has never been reported. Corroborating with Schaefer et al., it was observed herein that IL-10 KO BMPAC express significant upregulation of miR-375 at basal level and the levels of miR-375 are significantly upregulated in WT-BMPACs both under inflammation, hypoxia or ischemia. Interestingly exogenous IL-10 therapy significantly reduced miR-375 expression; suggesting IL-10 regulates miR-375 levels in BMPAC. Most importantly, miR-375 was identified as an IL-10-regulated miRNA and IL-10 directly limits miR-375 at a post-transcriptional level and thereby reducing its expression. The present data suggests miR 375 as a downstream target of IL-10 and that IL-10 mediated inhibition of miR 375 may in part explain positive effects of IL-10 in BMPACs.

It was confirmed that miR-375 directly interacts with PDK-1 by luciferase assay. This is consistent with a recent report showing that miR-375 directly targets PDK-1 at the protein level in gastric carcinoma cells (Yan et al., 2014, Int J Cancer 135:1011-8). Furthermore, knockdown of miR-375 increased the phosphorylation of Akt in BMPACs and post-MI heart by targeting PDK-1. Therefore, it is contemplated herein that repression of miR-375 may provide a survival advantage to BMPAC and cardiomyocytes via activation of the PDK-1/Akt survival pathway. Several reports suggest the importance of PDK-1 in cardiovascular biology. PDK-1-MCKCre mice showed impairment of LV contraction (Ito et al., 2009, PNAS 106:8689-94). It was also reported that cardiomyocytes deficient for PDK-1 were sensitive to hypoxia (Mora et al., 2003, EMBO J 22:4666-76), and that ischemic preconditioning failed to protect PDK-1-hypomorphic mutant mice against MI (Budas et al., 2006, FASEB J 20:2556-8). PDK-1 has been shown to be a pivotal effector to promote survival of cardiomyocytes in vivo (Ito et al., 2009, PNAS 106:8689-94). Therefore, it appears upregulation of PDK-1, by targeting miR-375 inhibition, in the hearts may emerge as a potential therapeutic strategy for heart failure.

Furthermore, the data presented herein shows that anti-miR-375 treatment enhances BMPAC functions such as tube formation ability, proliferation, and reduction in apoptosis in vitro and transplantation of anti-miR-375 treated BMPAC enhanced neovascularization, reduced infarct size, and attenuated LV dysfunction. Interestingly, miR-375 knockdown in IL-10 KO BMPAC partially restored their functions in vitro and in vivo as well, suggesting the observed functional benefits were IL-10 dependent. The data demonstrating increased PDK-1 expression and AKT phosphorylation in miR-375 knockdown BMPACs and the ischemic myocardium suggests the role of PDK-1 in enhancing BMPAC functional benefits. PDK-1 plays an important role in promoting cell survival, as loss of PDK-1 has been implicated in endothelial cell apoptosis. Therefore, the poorer tube formation, cell proliferation, and enhanced cell death of IL-10 KO BMPAC might be due to inactivation of AKT, which is well established to play an important role in endothelial cell biology and angiogenesis by activating anti-apoptotic, pro-survival signaling cascades (Friedrich et al., 2004, Mol Cell Biol 24:8134-44; Papapetropoulos et al., 2000, J Biol Chem 275:9102-5). The PDK-1 knockdown experiments in this study further confirm its crucial role in modulating BMPAC functions.

Another explanation for this finding could be the observation of enhanced paracrine factors secretion by anti-miR-375 treated BMPAC compared with scrambled control BMPACs contributing to not only vasculogenesis but also myocytes protection are in line with a recent report suggesting the role of miR-133a in enhancing CPCs paracrine effects (Izarra et al., 204, Stem cell Rep 3:1029-42).

These observations demonstrate that inhibition of IL-10 dependent miR-375 exhibits pleiotropic beneficial effects, which contribute to the enhanced BMPAC survival and angiogenic potential. Thus, miR-375 knockdown BMPAC therapy appears to be feasible approach to limit ischemic injury and might prove to be an attractive therapeutic strategy for patients with MI.

### Example 2: Knockdown of miR-375 Attenuates Post MI Inflammatory Response and Left Ventricular Dysfunction

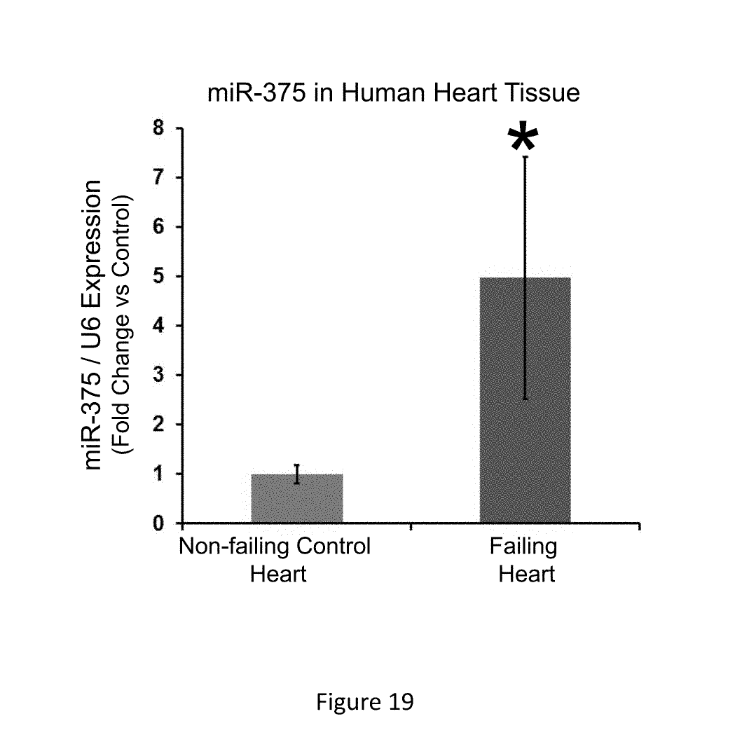

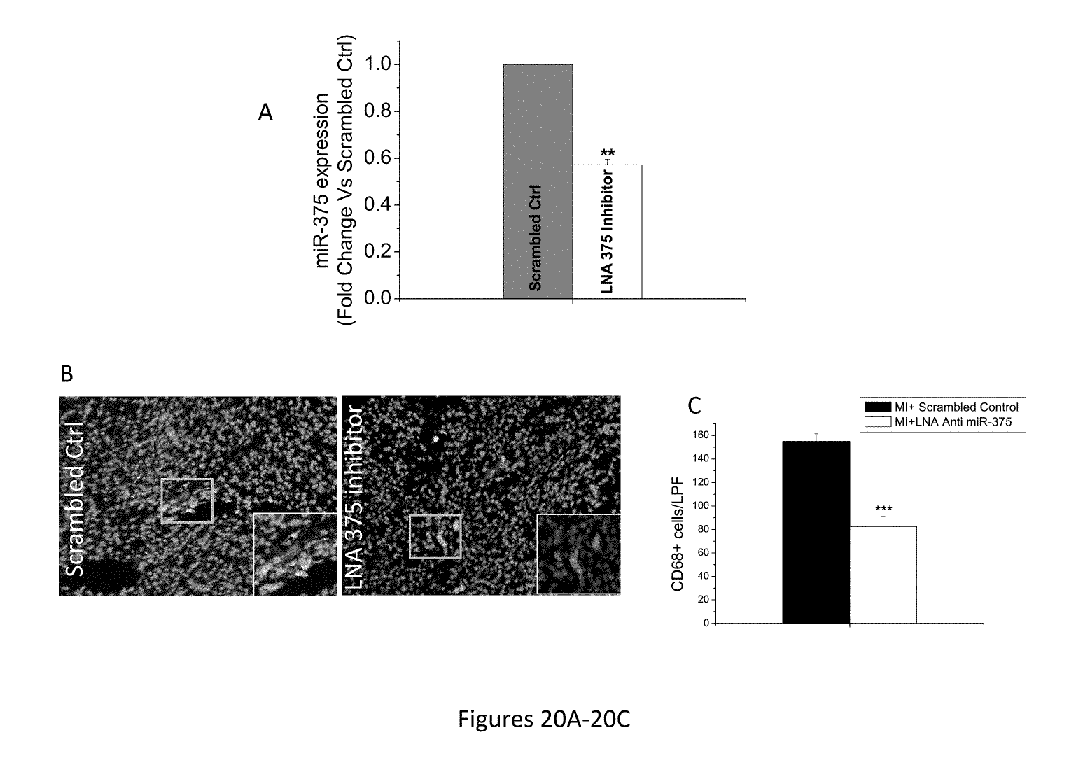

The results presented herein demonstrate that miR-375 is significantly increased in human heart failure patients (FIG. 19). miR-375 inhibitor locked nucleic acid 375 (LNA375) decreases miR-375 expression, inflammatory cells and infiltrating CD68⁺ cells in border zone of LV infarct at 5 d post-MI (FIG. 20).

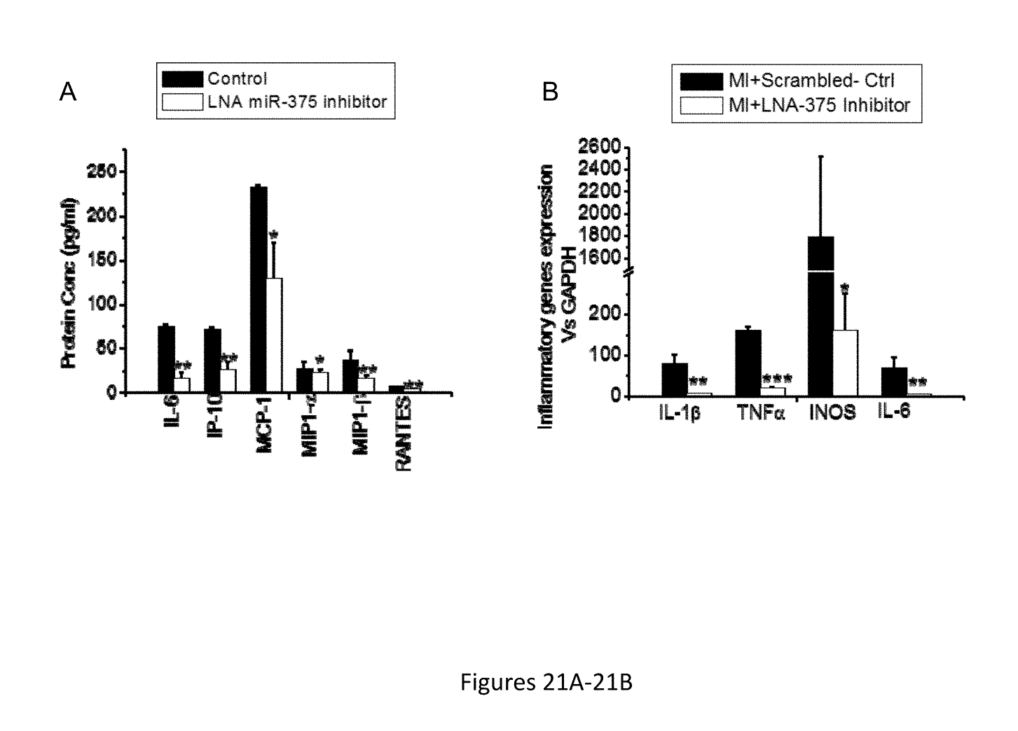

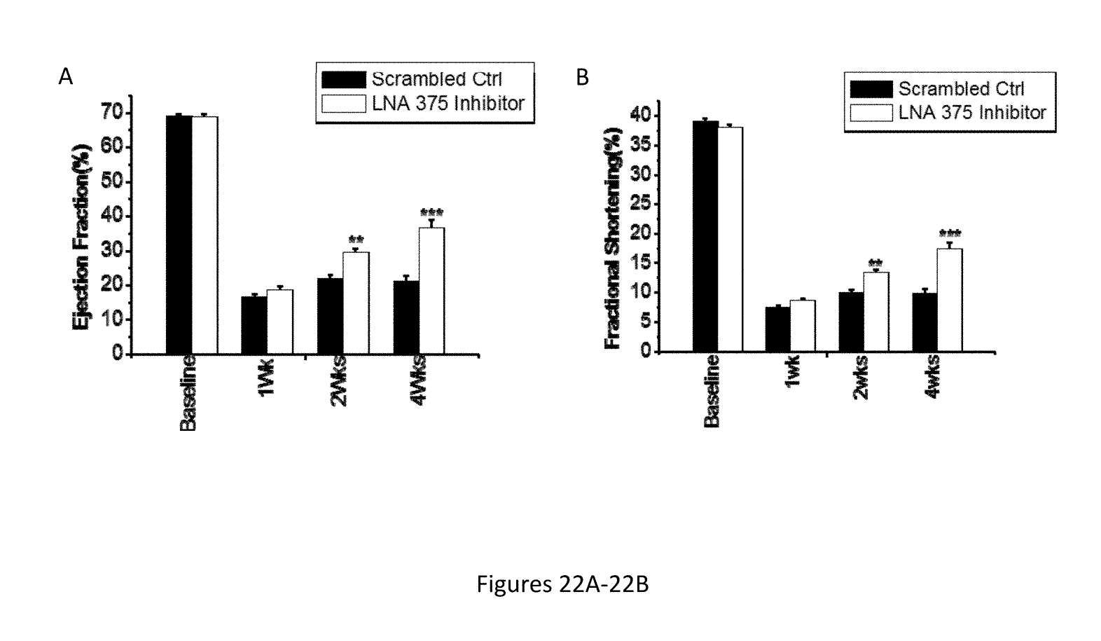

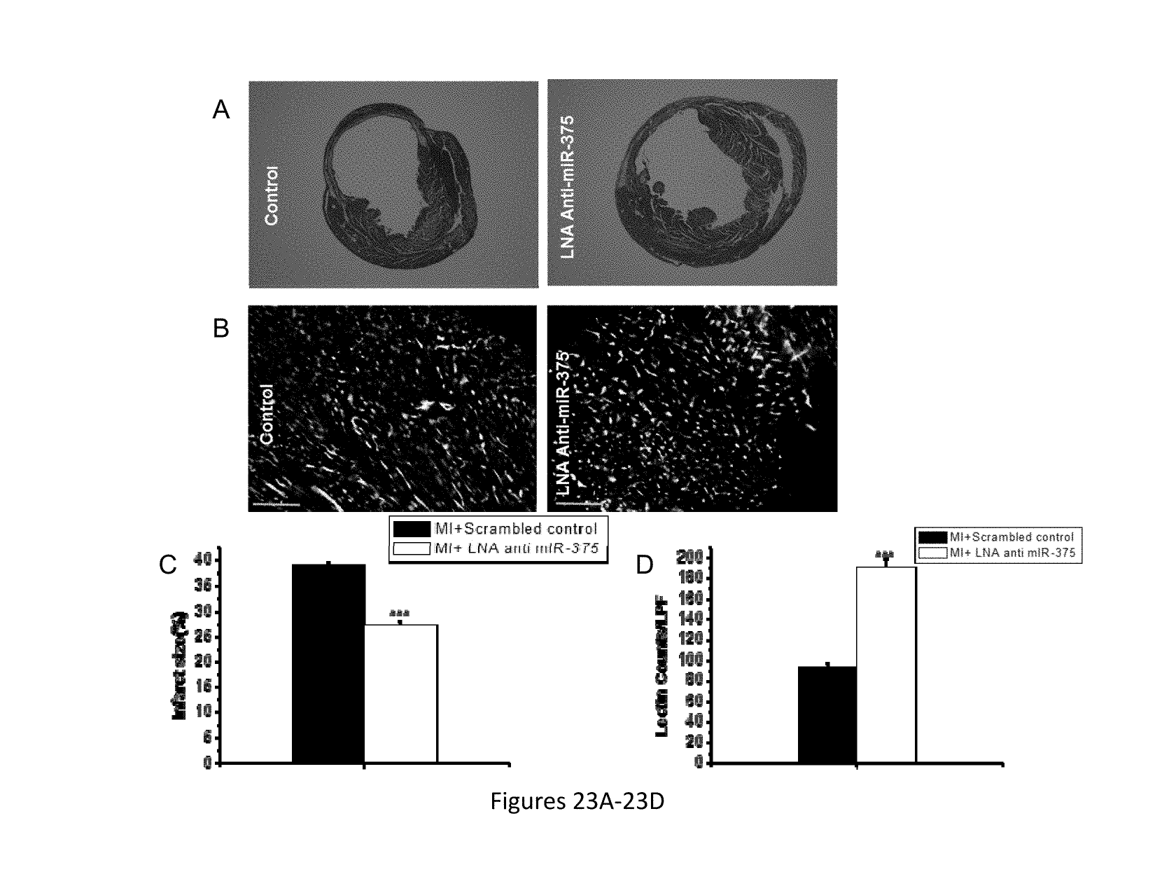

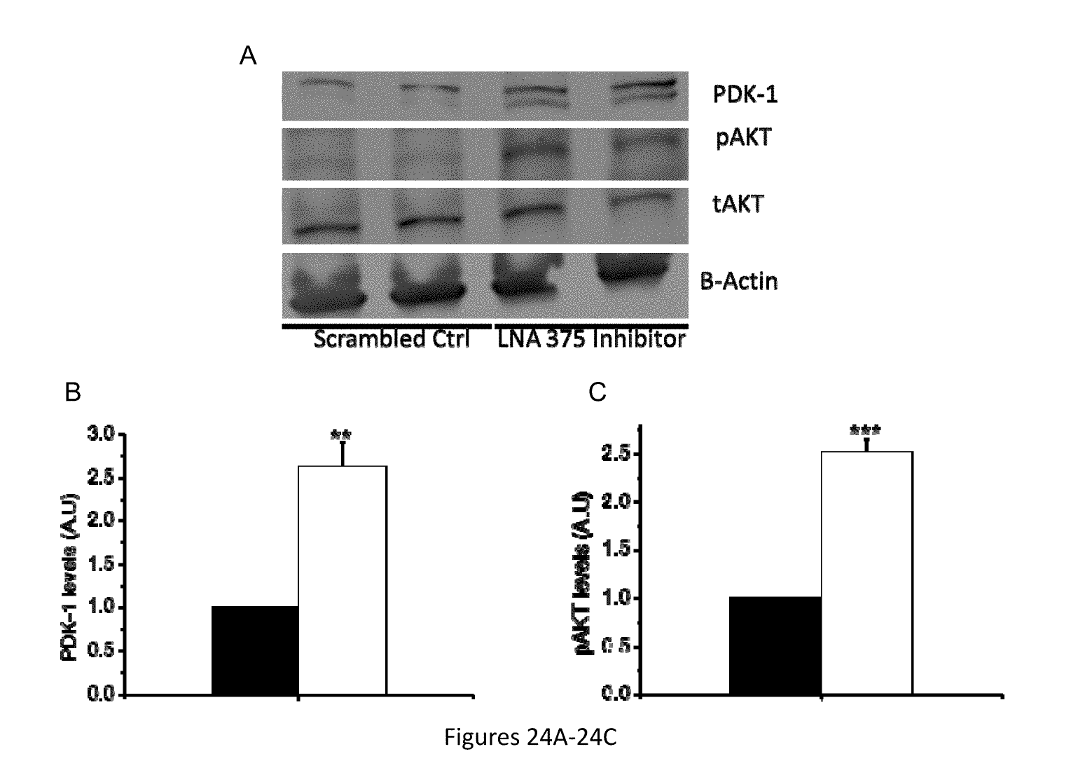

Further, LNA375 inhibit post MI inflammatory cytokine expression including IL-6, IP-10, MCP-1, MIP1α, MIP1β, and RANTES protein expression (FIG. 21A) and IL-1β, TNFα, iNOS, and IL-6 gene expression (FIG. 21B). Echocardiography analysis revealed LNA375 mediated myocardial miR-375 knockdown attenuates post-MI LV dysfunction (FIG. 22). LNA375 mediated myocardial miR-375 also reduces fibrosis (FIGS. 23A, 23B) and enhances neovascularization (FIGS. 23C, 23D) after MI. LNA375 also targets PDK-1 in the MI heart (FIG. 24). These results suggest that LNA375 may be used to inhibit miR-375 to treat ischemic injury due to myocardial infarction.

What is claimed is:

1. A composition for treating ischemic injury comprising an inhibitor of microRNA (miR)-375.

2. The composition of claim 1, wherein the inhibitor of miR-375 is an oligonucleotide.

3. The composition of claim 2, wherein the oligonucleotide comprises at least one locked nucleic acid.

4. The composition of claim 3, wherein the oligonucleotide comprises SEQ ID NO. 1

5. The composition of claim 2, wherein the oligonucleotide comprises SEQ ID NO. 2

6. The composition of claim 1, wherein the composition further comprises IL-10.

7. The composition of claim 1, wherein the composition further comprises a pharmaceutically acceptable carrier.

8. A method of treating an ischemic heart in a subject, the method comprising:
isolating a stem cell from a subject;
inhibiting miR-375 in the isolated stem cell to generate an miR-375 inhibited stem cell; and
administering the miR-375 inhibited stem cell to the subject.

9. The method of claim 8, wherein the stem cell is a bone marrow-derived angiogenic progenitor cell (BMAPC).

10. The method of claim 8, wherein inhibiting miR-375 in the stem cell further comprises administering to the cell an effective amount of an inhibitor of miR-375.

11. The method of claim 10, wherein the inhibitor of miR-375 comprises an oligonucleotide.

12. The method of claim 11, wherein the oligonucleotide comprises SEQ ID NO. 2.

13. The method of claim 11, wherein the oligonucleotide comprises at least one locked nucleic acid.

14. The method of claim 13, wherein the oligonucleotide comprises SEQ ID NO. 1.

15. The method of claim 8, wherein the method further comprises administering to the subject an effective amount of IL-10.

16. The method of claim 8, wherein the subject is a mammal.

17. The method of claim 8, wherein the subject is a human.

18. The method of claim 8, wherein administering the stem cell to the subject further comprises administering the stem cell to the subject through a route, the route selected from the group consisting of parenteral, intravenous, intraperitoneal, and bolus injection to a target tissue.

19. The method of claim 18, wherein the target tissue is a cardiac tissue.

20. A method of enhancing cell survival, the method comprising administering to the cell an effective amount of an inhibitor of miR-375.
